(19) United States
(12) Patent Application Publication
(10) Pub. No.: US 2022/0193642 A1
(43) Pub. Date: Jun. 23, 2022

(54) CATALYTIC PLASMONIC NANOMATERIAL

(71) Applicant: Habib Technologies LLC, Lancaster, PA (US)

(72) Inventors: Youssef M. HABIB, Lancaster, PA (US); Abraham ULMAN, Brooklyn, NY (US)

(21) Appl. No.: 17/428,794

(22) PCT Filed: Feb. 25, 2020
(86) PCT No.: PCT/US20/19655

§ 371 Date: Aug. 5, 2021

Related U.S. Application Data
(60) Provisional application No. 62/809,966, filed on Feb. 25, 2019.

(51) Int. Cl. B01J 23/89; B01J 21/06; B01J 23/50; B01J 21/02; B01J 35/00; B01J 35/04; B01J 37/34; B01J 37/02; B01J 15/00; B01J 19/12; B01J 19/00; C01C 1/04; C07C 31/04; C07C 29/157

(52) U.S. Cl. CPC B01J 23/8926; B01J 2219/0875; B01J 23/50; B01J 21/02; B01J 35/004; B01J 35/0013; B01J 35/04; B01J 35/0033; B01J 37/348; B01J 37/0225; B01J 37/0244; B01J 37/0228; B01J 15/00; B01J 19/127; B01J 19/006; C01C 1/0417; C01C 1/0411; C07C 31/04; C07C 29/157; B01J 2219/00763; B01J 2219/1203; B01J 2219/0892; B01J 21/063

(57) ABSTRACT

A method for producing plasmonic nanomaterials that are catalytically or photocatalytically active by fabricating plasmonic nanostructures on substrates using electrodeposition into a nano-template structure and forming a plurality of nanorods in an array, wherein the nanorods are made from materials chosen from the group consisting of materials that are plasmonic and/or catalytic, and materials that are catalytically activated by depositing pure elemental metals, alloys, or alternating layers of different metals or alloys, and producing catalytic plasmonic nanomaterials. Catalytic plasmonic nanomaterials made from the above method. An optical reactor device that utilizes catalytic nanomaterials for photocatalytic synthesis of methanol or ammonia. A method of photocatalytic synthesis of methanol and ammonia by using catalytic plasmonic nanomaterial to convert CO₂ and H₂ to methanol and N₂ and H₂ to ammonia using optical power. A hybrid plasma-plasmonic reactor for the utilization of CO₂ and CH₄ to produce methanol, ethylene, and acetic acid.

### GRANT INFORMATION

Research in this application was supported in part by grants from the US Department of Energy Chicago Office and the US Department of Energy Office of Science (Grant No. DE-SC0015942 and DE-SC0019657). The Government has certain rights in the invention.

### BACKGROUND OF THE INVENTION

### 1. Technical Field

The present invention relates to plasmonic nanostructures. More specifically, methodologies for the fabrication and manufacture of catalytically active plasmonic nanostructures are disclosed, as are techniques to utilize these nanomaterials for photocatalysis and chemical synthesis. The nanomaterial invention interacts with electromagnetic radiation (light) generating plasmonically-induced heat and energetic carriers (hot electrons and holes) that are utilized to facilitate chemical reactions. The invention is in the technical field of fabricating catalytic plasmonic nanomaterials and applications thereof.

### 2. Background Art

According to the U.S. Department of Energy (DOE), catalysts are used in 90% of U.S. chemical manufacturing processes and in making over 20% of all industrial products. A catalyst is a substance that increases the rate of a chemical reaction by lowering its activation energy, without being consumed in the process. Industrial chemicals are produced in vast quantities using catalytic processes, which are also employed in producing smaller amounts of high-value specialty chemicals and pharmaceuticals.

Methanol, or methyl alcohol, is primarily produced today by the catalytic hydrogenation of carbon monoxide sourced from synthesis gas. In 2014, global methanol production was 75 million metric tons, which should increase to 133 million metric tons by 2020. Methanol was a $55 Billion global industry in 2015. The market for catalytic materials used in global methanol production was $288.7 million in 2015. Methanol is used in the manufacture of many consumer products and is a widely used fuel source. Methanol is a feedstock to produce numerous chemicals such as acetic acid (About 75% of acetic acid made for use in the chemical industry is made by the carbonylation of methanol) and formaldehyde, which in turn are used in products like adhesives, foams, plywood subfloors, solvents and windshield washer fluid.

While the International Renewable Energy Agency (IRENA) claim that the share of renewable energy in the power sector would increase from 25% in 2017 to 85% by 2050 primarily through growth in solar and wind power generation, with a concurrent reduction of energy-related CO₂ emissions, the Department of Energy projections to 2050 state that fossil fuels are the energy sources of America's future. Many studies lack discussion about using CO₂ as a feedstock for fuel production. CO₂ utilization can be accomplished by reacting with other sources; including hydrogen (H₂), water (H₂O), and methane (CH₄). Methane is the second largest (16%) contributor to greenhouse gas emissions after CO₂. Recycling CO₂ emitted by power stations to synthesize fuels and valuable chemicals represents a new paradigm in energy use and reuse. The invention is an enabling material for the realization of catalytic CO₂ recycling through the targeted synthesis on methanol (CH₃OH), other oxygenates like acetic acid (CH₃COOH), formaldehyde (HCHO), and ethanol (C₂H₅OH) as well as hydrocarbons like acetylene (C₂H₂), ethylene (C₂H₄), and ethane (C₂H₆), and other industrial catalytic processes like the production of ammonia (NH₃) by combining nitrogen (N₂) with a hydrogen source such as hydrogen (H₂), water (H₂O), or methane (CH₄).

Methods for carbon capture and utilization present both business and environmental opportunities in scenarios where industrial CO₂ is stored and used as a raw material for the synthesis of fuels, chemicals, and other resources. The hydrogenation of carbon dioxide to methanol is an attractive route in this regard, as methanol can be utilized as a fuel, a vehicle for hydrogen storage, and a constituent in the synthesis of olefins. Currently, the process of synthesizing methanol from captured CO₂ and sourced H₂ is performed under catalytic reaction conditions employing high-pressures and temperatures requiring sophisticated process equipment that consumes large amounts of energy. The largest such system is the George Olah plant in Iceland that harnesses geothermal power to generate over 5 million liters of methanol annually, while consuming over 5500 tons of CO₂ in the process. A rigorous lifecycle analysis of the impact and cost of industrial methanol synthesis from CO₂ feedstock in these production scale systems concluded that while the present technologies do lead to a significant net CO₂ emission reduction; they are not financially viable as currently implemented. The effectiveness depends highly on the sourcing of both input gases as well as the reactor power. Overcoming the financial constraints of adopting CO₂ derived methanol as an abundant fuel source with a negative carbon footprint requires the development of advanced catalytic materials that decrease activation energies and employ novel reactor designs powered by renewable energy sources.

The limiting factor in current reactors is the activation energy required to reduce carbon dioxide. Traditionally, thermal energy is applied to a vessel containing the catalyst and reactants that operates at a temperature and pressure suitable to promote the desired reaction. In the case of CH₃OH formation from CO₂ and H₂, promoting the forward reaction while limiting the reverse is crucial and requires costly, multi-stage reaction vessels operating at threshold pressures>40 Bar and temperatures>200° C. Innovative catalytic materials that utilize unconventional energy sources and reaction methods are fundamental to making the synthesis of CH₃OH from CO₂ and hydrogen sources such as hydrogen (H₂), water (H₂O), or methane (CH₄) an economically viable reality.

Ammonia (NH₃) was produced by 13 companies at 31 plants in 15 States in the United States during 2016. The United States is one of the world's leading producers and consumers of ammonia and derives urea, ammonium nitrate, ammonium phosphates, nitric acid, and ammonium sulfate from it. Estimated production in 2017 was over 150 million tons, and capacity is estimated at over 230 million tons. Global production is expected to continue to grow by 3-5%/year.

The Haber-Bosch process, from its conception in 1908, and immediate translation into large-scale production in 1913, has been the preferred method for the synthesis of ammonia. Ammonia is produced in large quantities using N₂ harvested from air, and H₂ harvested from steam reformation of hydrocarbons, and the water-gas shift reaction of CO with H₂O from steam or air and utilizes iron catalysts iron promoted with K₂O, CaO, SiO₂, and Al₂O₃. However, the high-temperature (˜400-500° C.) and high-pressure (˜150-250 Bar) requirements of the process make it energetically highly inefficient. Consequently, energy consumption from ammonia production is the largest in the chemical industry and CO₂ emissions are at least 2× the production volume. In total Haber-Bosch processes consume about 5% of the world's natural gas and comprise ˜2% of global energy usage.

Catalytic plasmonic nanostructures can be used for innovative photocatalytic synthesis of important chemical and fuels by effectively lowering the activation energy of the process. When metallic nanostructures are illuminated with radiation near the resonant wavelength, collective electron excitations (plasmons) are induced within their sub-wavelength dimensions, resulting in a localized direct energy transfer process that produces heat and high-energy “hot” electrons that interact with adsorbates and promote catalytic chemical processes. The nanostructure surface becomes thermally and chemically catalytically active in the chemical transformation of molecules adsorbed by reducing the activation energy leading to the synthesis of higher order molecular species. The surfaces can be engineered to interact optimally with specific adsorbates. For example, plasmonic nanostructures can trigger facile reductive dissociation of adsorbed H₂ molecules, and consecutive reactions of the hydride and the hydrogen atom with CO₂ or other molecules can take place in the pathway of CO₂ reduction to produce methanol (CH₃OH) or even methane (CH₄). Halas, et al. demonstrated light-driven plasmonic photocatalysis to convert CO₂ into CO at significantly milder operating conditions than its thermally activated counterpart, and demonstrated that the plasmon-induced, carrier-driven reaction occurs at a higher rate and lower temperature (175° C. vs 400° C.) than the thermally driven one. Plasmonic materials provide a mechanism for light-activated photocatalysis that lowers activation energies by injecting high-energy electrons (hot-carriers) into adsorbates.

A bimetallic catalyst is comprised of two metals that are separately active for a given chemical system, but together at differing ratios can display a synergistic effect leading to enhanced activity and a more effective result. For example, Cu—Pd bimetallic catalysts act together on H₂ and CO₂ to facilitate the breaking of chemical bonds at lower energies than without the catalyst which enhances CH₃OH formation rates over either Cu, or Pd monometallic catalysts as the two metals act in a bifunctional manner to promote the desired reaction. Various combinations of metals can be used in making plasmonic catalyst materials by exploiting the plasmonic and catalytic properties of individual materials in combinations such as core-shell, layered, and alloyed nanostructured morphologies.

Other examples of uses of plasmonic nanomaterials include CA 3062848 to Halas, which discloses a method of making a multicomponent photocatalyst, includes inducing precipitation from a pre-cursor solution comprising a pre-cursor of a plasmonic material and a pre-cursor of a reactive component to form co-precipitated particles; collecting the co-precipitated particles; and annealing the co-precipitated particles to form the multicomponent photocatalyst comprising a reactive component optically, thermally, or electronically coupled to a plasmonic material.

U.S. Pat. No. 9,815,702 to Kuhn discloses systems and methods for converting carbon dioxide into useful chemical feedstock, such as carbon monoxide, which can be used in industrial processes including fuel synthesis and the production of carbon fiber products. Carbon dioxide from a source, such as a power plant, is passed through catalyst material that removes oxygen atoms from the carbon dioxide molecules to form carbon monoxide. The catalyst material is an intimate mixture of oxygen-conducting material and plasmonic material that absorbs solar energy. In such cases, the heat required for the reaction can be obtained from the solar energy.

There remains a need for plasmonic nanomaterials that are useful as photocatalytic platforms for the synthesis of methanol, ammonia, and other important chemicals and fuels.

### SUMMARY OF THE INVENTION

The present invention provides for a method for producing plasmonic nanomaterials that are catalytically or photocatalytically active by fabricating plasmonic nanostructures on substrates using electrodeposition into a nano-template structure and forming a plurality of nanorods in an array, wherein the nanorods are made from materials chosen from the group consisting of materials that are plasmonic and/or catalytic, and materials that are catalytically activated by depositing pure elemental metals, alloys, or alternating layers of different metals or alloys, and producing catalytic plasmonic nanomaterials.

The present invention provides for catalytic plasmonic nanomaterials made from the above method.

The present invention provides for an optical reactor device that utilizes plasmonic catalytic nanomaterials for photocatalytic synthesis of fuels and chemicals including oxygenates like methanol, hydrocarbons like ethylene, or non-carbon compounds like ammonia, including a chemical reaction chamber containing catalytic plasmonic nanomaterial, the chemical reaction chamber including a gas distribution manifold for flowing gas containing reactive components over the catalytic plasmonic nanomaterial and a gas collection manifold for collecting synthesized gas products, wherein the chemical reaction chamber includes a mechanism of providing optical energy to the catalytic plasmonic nanomaterial through illumination and provides constant temperature control of the chemical reaction chamber.

The present invention also provides for a method of photocatalytic synthesis of chemicals and fuels including methanol and ammonia by using catalytic plasmonic nanomaterial to convert CO₂ and H₂ to methanol and N₂ and H₂ to ammonia using optical power.

The present invention provides for methanol and ammonia made by the above method.

The present invention provides for a method of synthesizing useful chemicals from greenhouse gases such as CO₂ and CH₄ waste gases, and stable molecules that are used as sources in the synthesis of chemicals and fuels by using catalytic plasmonic nanomaterial to convert greenhouse gases to useful chemicals using optical power.

The present invention further provides for a method of making plasmonic nanomaterial, by forming plasmonic nanorods on a flexible substrate.

The present invention provides for a method of producing chemicals by stimulating plasmons in catalytic plasmonic nanomaterials with photons in a plasma catalytic reactor and producing chemicals.

The present invention provides for a hybrid plasma-plasmonic reactor device that utilizes plasmonic catalytic nanorod arrays for synthesis of fuels and chemicals including methanol or ammonia, including a reaction chamber containing a first adjustable disc electrode having first catalytic plasmonic nanomaterial layer thereon and a second adjustable disc electrode having a second catalytic plasmonic nanomaterial layer thereon, the reaction chamber including a gas inlet for flowing gas containing reactive components over the first and second catalytic plasmonic nanomaterials and a gas outlet for collecting synthesized gas products, wherein the first and second catalytic plasmonic nanomaterial layers ignite a plasma from gas introduced into the reaction chamber and synthesize fuels and chemicals.

### DESCRIPTION OF THE DRAWINGS

Other advantages of the present invention are readily appreciated as the same becomes better understood by reference to the following detailed description when considered in connection with the accompanying drawings wherein:

FIG. 1 is a representation of the plasmonic nanomaterial invention as produced in a roll-to-roll format on a flexible substrate;

FIGS. 2A-2D are representational schematics showing the four-step fabrication process used to produce plasmonic nanorod arrays: FIG. 2A is a representation of the plasmonic nanomaterial precursor material showing substrate, conductive seed layer and aluminum (Al) process layer, FIG. 2B is a representation where the Al layer was anodized to create a nanoporous anodic aluminum oxide (AAO) template layer with pores penetrating through to the seed layer, FIG. 2C is a representation after electro-deposition of material into the AAO template to form nanorods, and FIG. 2D is a representation of an exposed nanorod array after AAO template layer removal;

FIGS. 3A-3D show four successively higher magnification Scanning Electron Microscope (SEM) images of the nanomaterial processed to Step 2B showing a structured and ordered AAO layer: FIG. 3A has a 2-micron scale, FIG. 3B has a 1 micron scale, FIG. 3C has a 300 nm scale, and FIG. 3D has a 100 nm scale;

FIGS. 4A-4C show three successively higher magnification SEM images of the plasmonic nanomaterial processed to Step 2C showing long range order and continuity of nanorods formed within the AAO matrix: FIG. 4A has a 200 nm scale, FIG. 4B has a 100 nm scale, and FIG. 4C has a 100 nm scale;

FIGS. 5A-5D show four successive Scanning Electron Microscope (SEM) images of the nanomaterial processed to Step 2D showing the showing long range order and continuity of an exposed nanorod array: FIG. 5A has a 1-micron scale, FIG. 5B has a 200 nm scale, FIG. 5C has a 200 nm scale, and FIG. 5D has a 100 nm scale;

FIG. 6A is a characteristic anodization current versus time plot used in converting Al into AAO in the plasmonic nanomaterial fabrication process, and FIG. 6B is a characteristic electrodeposition current versus time plot used to control and monitor the stages of the nanorod array fabrication process;

FIGS. 7A-7C show three SEM images of the plasmonic nanomaterial invention fabricated with increasing nanorod lengths: FIG. 7A shows an SEM image of silver nanorods with an average length of 270 nm, FIG. 7B shows an SEM image of silver nanorods with an average length of 355 nm, and FIG. 7C shows an SEM image of silver nanorods with an average length of 488 nm;

FIGS. 8A-8C show three Scanning Electron Microscope (SEM) images of the plasmonic nanomaterial with different nanorod pitch, the nanorods were grown under 70V, 80V, and 90V constant anodization potentials in the same electrolyte, pitch distances increased from 150 nm in FIG. 8A to 200 nm in FIG. 8C, the scale bars represent 100 nm for all;

FIG. 9 is a representation of three plasmonic nanorods: the center shows a bare metallic nanorod, on the right is one with a bimetallic catalytic coating applied to the surface, while the image on the left shows a nanorod with a catalytic coating applied by physical vapor deposition (sputter coating).

FIG. 10 is a schematic representation of the present catalytic plasmonic nanomaterial invention showing an array of metallic nanorods fabricated on glass with a bimetallic layer coating the surface;

FIG. 11 is a graph of optical absorbance vs wavelength spectral data showing plasmon resonance from representative gold and silver plasmonic nanorod arrays as fabricated in the present invention;

FIG. 12A shows the chemical equation for the synthesis of methanol from carbon dioxide and hydrogen as performed in the present invention, FIG. 12B shows the chemical equation for the synthesis of ammonia from nitrogen and hydrogen as performed in the present invention, and FIG. 12C shows the molecular structure of a catalytic organo-metallic compound with phosphonic acid attached for the attachment to silver nanorods in the present invention;

FIG. 13A is a schematic representation of layered catalytic and/or plasmonic nanorods fabricated from two materials, FIG. 13B is an SEM image of a two-layer gold/silver plasmonic nanorod array, FIG. 11C is an SEM image of a five-layer gold/silver plasmonic nanorod array with color enhancement on one nanorod;

FIG. 14 is a graph of optical absorbance versus wavelength spectral data from FIG. 11 with data from the two- and five-layer gold/silver layered plasmonic nanorod arrays from FIGS. 13A-13B added to the graph;

FIG. 15 is a schematic representation of an optical flow reactor employing the catalytic plasmonic nanomaterial invention for photocatalytic chemical synthesis;

FIGS. 16A-16C are perspective views of a solar powered optical flow reactor design that utilizes the catalytic plasmonic nanomaterial invention: FIG. 16A shows a schematic of the reaction tube with the catalytic plasmonic nanomaterial inside, FIG. 16B is a representation of the reaction tube inserted into a solar concentrating parabolic reflector, and FIG. 16C shows details of how the catalytic plasmonic nanomaterial invention is inserted into the reactor tube with gas flow manifolds situated at either end;

FIG. 17A is a side perspective view of a liquid immersion electrochemical process cell for fabricating the present nanomaterial invention by batch production, and FIG. 17B is a photograph of a catalytic plasmonic nanomaterial sample as fabricated on a 5 cm×5 cm WILLOW® glass coupon using the process cell;

FIG. 18 is a schematic of the two plasmonic phenomenon exploited for photocatalysis wherein the absorption of light results in very high temperatures localized in the nanorod, and the presence of high-energy charges that are used to facilitate chemical reactions;

FIG. 19A is an energy dispersive x-ray (EDX) compositional analysis data map of plasmonic catalytic nanomaterial, and FIG. 19B is a table of the EDX compositional analysis data;

FIG. 20A-20D are graphs of x-ray photoemission spectroscopy (XPS) data showing surface states of Cu and Pd sputter coated silver nanorods, FIG. 20A is a survey graph, FIG. 20B is a graph of Cu, FIG. 20C is a graph of Pd, and FIG. 20D is a graph of Ag;

FIG. 21A is a graph of XPS data for Ag, FIG. 21B is a graph of XPS data for Pd, and FIG. 21C is a graph of XPS data for Cu performed on electrolessly deposited Cu and Pd on silver nanorods;

FIGS. 22A-22C are SEM images of nanorods FIG. 22A is pure silver nanorods, FIG. 22B is silver nanorods with Cu and Pd bimetallic sputter coating layer, and FIG. 22C is silver nanorods with electrolessly deposited Cu and Pd bimetallic layer;

FIG. 23 is a graph comparing the UV-Vis spectra for silver nanorods as produced, with a Cu and Pd sputter coated layer, and an electrolessly deposited Cu and PD layer;

FIG. 24 is a photograph of a custom built photoreactor for photocatalytic synthesis;

FIG. 25 is a table showing the different samples tested in a batch reactor that marks the conditions where enhanced reaction rates were observed under illumination;

FIG. 26 is a graph of kinetic reaction data from the reactor for samples under dark and illuminated conditions showing optical enhancement with light;

FIGS. 27A-27D are graphs of products obtained via plasmonic photocatalysis from CO₂ and H₂ under batch process conditions, showing ppm of methanol (FIG. 27A), carbon dioxide (FIG. 27B), carbon monoxide (FIG. 27C), and methane (FIG. 27D);

FIG. 28 is a graph of illumination dependence of optical CO production from CO₂ at two temperatures;

FIG. 29 is a matrix of metals for use in the present nanomaterial invention ranking their relevant properties;

FIGS. 30A-30H are a depiction of four nanorod structural morphologies in top down planar and cross-sectional perspectives; FIGS. 30A and 30E are pure metal, FIGS. 30B and 30F are a core shell (antenna reactor) geometry, FIGS. 30C and 30G are a layered (antenna reactor) structure, and FIGS. 30D and 30H are an alloy or bimetal;

FIGS. 31A and 31B are side views of optical tube reactors with baffles of catalytic plasmonic nanomaterial inside, FIG. 31C is a reactor with a solar concentrator r, and FIG. 31D is a large array of reactors and solar collectors;

FIG. 32A is a depiction of tilted catalytic plasmonic nanomaterials photoreactor insert for increased reaction area and light harvesting, FIG. 32B is a depiction of curved catalytic plasmonic nanomaterials photoreactor insert for increased reaction area and light harvesting, and FIG. 32C is a depiction of a baffled reactor that extends the residence time of reactants in the chamber;

FIG. 33 is a dielectric barrier discharge (DBD) plasma reactor design that couples plasma energy into catalytic plasmonic nanomaterials; and

FIG. 34 is a schematic representation of conditions inside a hybrid plasma plasmonic catalytic reactor system synthesizing methanol from CO₂ and CH₄.

### DETAILED DESCRIPTION OF THE INVENTION

The present invention provides for catalytic plasmonic nanomaterials and methods for the fabrication of such catalytic plasmonic nanomaterials. The present invention further provides reactor designs and methods for synthesizing methanol, ammonia, and other chemicals, both gaseous and liquid products, including pharmaceuticals by irradiation the catalytic plasmonic nanomaterials in a reactor. The present invention can take stable molecules including greenhouse gases like carbon dioxide and methane or abundant yet hard to react species like water and use them as sources for the catalytic synthesis of useful chemicals and fuels.

Most generally, the present invention provides for a method of producing plasmonic nanomaterials that are catalytically or photocatalytically active by fabricating plasmonic nanostructures on substrates using electrodeposition into a nano-template structure and forming a plurality of nanorods in an array, wherein the nanorods are made from materials chosen from the group consisting of materials that are both plasmonic and catalytic, and materials that are catalytically activated by depositing pure elemental metals, alloys, or alternating layers of different metals or alloys, and producing catalytic plasmonic nanomaterials.

Plasmonic nanomaterial includes a plurality of nanostructures attached to a substrate with the nanostructures specifically designed and intended to interact with optical energy (light) via plasmonic energy exchange. The catalytic nanomaterials of this invention include vertically aligned arrays of nanorods fabricated with densities of 10⁹ to 10¹¹ nanorods/cm², diameters of 25-900 nm and lengths of 0.1-10 microns. These nanoscale structures, also known as nanowires or nanobristles, are attached to the surface of a carrier, or substrate material that can be planar, cylindrical or otherwise. The nanorods have a cylindrical shape with one radial end of the cylinder attached to the substrate such that a multiplicity of nanorods arrayed on a surface can appear as a “bristled” surface. The bristled surface has a substantially enhanced surface area (2×-100×) than the substrate on which it is fabricated.

The nanorods are produced using electrochemical and chemical fabrication methods that allow precision control over the length, diameter, spacing, and material properties of the nanorods, which in turn determines their plasmonic properties and spectral response. The nanorods can vary in geometry from short, quasi-hemispherical low-aspect structures to elongated, high-aspect bristles with these geometrical variations affecting the optical response of the nanorods and providing a means to manipulate and control the optical characteristics of the material. By manipulating both the geometrical and material properties of the nanorods in the present invention, the optical response and catalytic action can be tuned to target the synthesis of particular chemicals or compounds.

The nanorods are plasmonic in that they are made of a material and in a geometrical size that supports a plasmon, surface plasmon, or plasmon resonance. An electromagnetic interaction between the nanorod and radiant energy (light) takes place where the light is absorbed by the nanorod(s) and the absorbed optical energy is manifested in the generation of a plasmon. The plasmon is a collective electron oscillation that dampens out on picosecond time scales resulting in the localized generation of heat and energetic charge carriers to interact with adsorbates on the nanorod surface.

A plasmonic nanomaterial is catalytic if its nanostructures are composed of or coated with a material that can promote catalytic or photocatalytic chemical reactions. The catalytic plasmonic nanomaterials can be formed as a ribbon, sheets, or rolls on the surfaces of flexible glass, metal foils that can include different layers, or polymeric materials; on the surface of threads or fibers produced from glass, polymers, or metals; or in a rigid planar design on both insulating or conducting substrates, or on the inner and/or outer surfaces of a tube. The catalytic nanomaterial transforms electromagnetic irradiation into a plasmon thus acting as an energy source to provide the Gibbs free energy for catalytic chemical reactions.

Chemical adsorbates on the surface of the nanostructures will undergo catalytic chemical transformation due to three properties of the nanomaterial: a) the chemical composition of the material comprising the nanostructure, b) heat generated locally in the subwavelength nanostructure by the plasmonic response, and c) high energy “hot electrons” and holes generated by the plasmon decay that promote alterations in chemical bonding and molecular structure. These features can act synergistically to reduce activation energies of processes such as oxidation or reduction.

The catalytic plasmonic nanomaterial can have various nanoscale formats employing a core shell or antenna reactor type geometry wherein arrays of plasmonic nanorods are catalytically activated by: i. By being coated or capped with another metal layer via vapor deposition, ii. By being coated or capped with various metal or conducting alloys or bimetallic layers via vapor deposition, iii. By being coated or capped with various metals, or conducting alloys, or bimetallic layers by electrochemical or electroless deposition. iv. Coated or covered with nanometer scale islands of metal, bimetals, or metal alloys via electroless chemical deposition, iv. Coated or covered with nanometer scale islands of metal, bimetals, or metal alloys via electrodeposition, v. Coated or capped with a semiconductor or metal oxide layer, and vi. Modified with chemically attached organometallic catalytic complexes resulting in heterogenization of homogeneous catalyst, vii. Made of a material or materials that are both plasmonic and catalytic in nature.

Further disclosed is the use of the catalytic nanomaterial in optical flow reactors, providing unique embodiments for chemical synthesis via catalytic reaction both in gas and in liquid phases, with facile and easy catalyst recovery and replacement by virtue of the nanomaterial being attached to a substrate.

The plasmonic nanomaterial is used for various forms of energy harvesting and transduction, including solar energy, optical energy, and plasma energy. It acts to convert electromagnetic energy directly and efficiently into heat and can be used for directly promoting phase transitions such as generating steam when illuminated in an aqueous environment. Modeling shows that the temperature of gold nanoparticles can be raised from room temperature to >795 K (522° C.) in just a few nanoseconds with a low light luminance, owing to enhanced light absorption through strong plasmonic resonance in structures subwavelength in dimension.

Plasmonic materials can effectively couple radiation into subwavelength sized metallic nanostructures that exploit electron oscillations excited through plasmon resonance decay non-radiatively, which lead to localized photothermal heating and the injection of high-energy hot electrons on the surface. The conversion of optical energy into heat and energetic charges is used to promote chemical reactions. Synchronous oscillations of the electron cloud within the nanostructures are stimulated by incident light. The dissipation or dephasing of the plasmon results in very high thermal energy density and the generation of hot electrons and holes within the nanostructures, and these properties can be used for photochemistry, photocatalysis, or photodetection. Hot electrons or holes can be excited by illuminating the material at the resonance energy which excites a continuum of energies through intra-band transitions, or off resonance that will excite inter-band excitations from a filled orbital to one that is unoccupied.

The plasmonic nanomaterial is fabricated using the process described in detail in U.S. Pat. No. 7,713,849 and application No. US 2018/0135850. It is formed on a substrate that is first coated with a thin film conductive layer (such as silver, or other metallic and conductive oxide materials such as Ag, Au, Cu, Co, Fe, W, Pd, Ni, ITO, AZO, etc.), followed by an Al metal layer is deposited by vapor deposition to produce the precursor material for nanofabrication. It can also be formed directly or indirectly on the surface of metal substrates, eliminating or reducing the need for coatings.

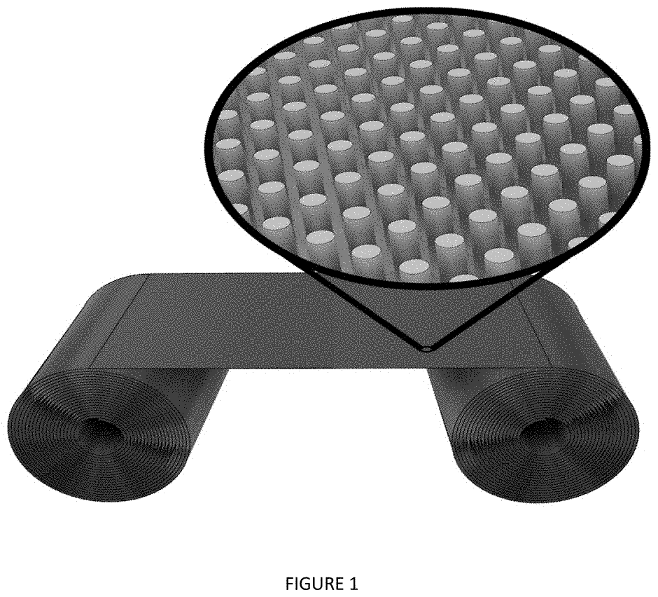

FIG. 1 shows a schematic representation of the plasmonic nanomaterial as manufactured continuously using a rolled substrate that for example can be flexible glass available from CORNING® as WILLOW® Glass product that is a 100 microns thick glass; a variety of polymers such as polyimide available from DUPONT® as KAPTON® or polyvinyl fluoride (PVF) available from DUPONT® as TEDLAR®; a variety of metal foils or metallic coated polymers; or advanced ultra-thin carriers such as those produced using graphene. While shown here fabricated continuously in a planar, sheet geometry, threads, fibers, foils and other spooled substrates can be equally employed. The nanomaterial is comprised of vertically aligned arrays of metallic nanorods robustly attached to the substrate surface and protruding from to form a plurality of bristle type structures. The nanorods are the active photocatalytic material structures that efficiently transduce optical energy within their subwavelength material dimensions through plasmon resonance. In general, when flexible substrates are used, the fabrication can be performed in a continuous or roll-to-roll format. When rigid substrates are used, batch processing can be used with an immersible electrochemical cell.

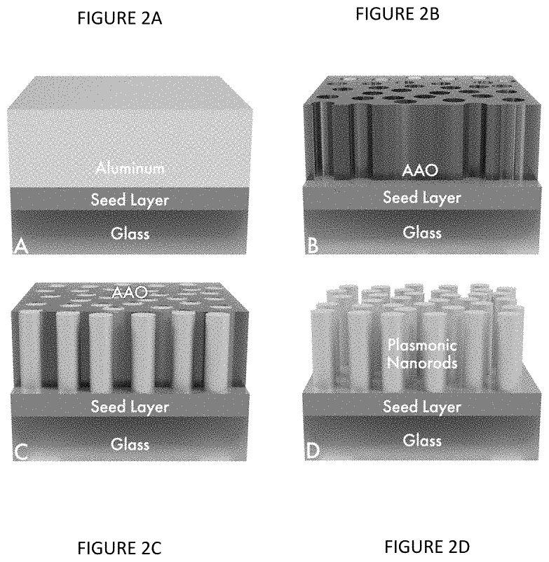

Referring now to FIGS. 2A-2D, this schematic representation shows the four-step nanofabrication process used to produce the plasmonic nanomaterial. In an embodiment of the four-step process for fabricating the present invention as presented in FIG. 2A, the starting substrate is a glass coupon that is coated with a silver conductive seed layer that is ˜50 nm thick using vacuum evaporative deposition system employing standard rf or dc magnetron sputter coating or electron beam based evaporative coating techniques. A thin (5-25 nm) layer of Ti or other bonding agent deposited in between the glass and conductive layer can be used as necessary to improve adhesion. Without breaking vacuum, an Al layer that is 100-1000 nm thick is then deposited on top of the conductive silver layer by physical vapor deposition methods such as electron beam evaporation, such that the three layers are adhered to each other and hence, the underlying substrate. Deposition techniques such as ion beam assisted electron beam evaporation is employed to minimize the formation of grains and grain boundaries and maximize the density of the materials as the thin films are being prepared. The conductive layer in FIG. 2A used in the device can be made from a number of materials including: a metal such as silver, gold, aluminum, tungsten, nickel, palladium, cobalt, molybdenum, platinum, copper, zinc, iron iridium, or many others; transparent conductive oxides such as indium tin oxide (ITO), aluminum-doped zinc oxide (AZO) and others; conducting polymers such as poly(3,4-ethylenedioxythiophene) (PEDOT); and carbon nanotube (fullerene) or graphene based conductive layers. Silver used in FIG. 2A provides robust adhesion to the underlying glass and contributes to the overall plasmonic optical response. The conductive layer's function is to provide electrical contact for anodization and to seed the nanorod growth for attachment to the substrate. Similarly, the Al layer in FIG. 2A can also be replaced by other metals that are amenable to anodization and the formation of a nanoporous oxide layer such as titanium (Ti), magnesium (Mg), niobium (Nb), tin (Sn), or cobalt (Co). Similarly, the process layer can be made of a material amenable to chemical conversion into a nanoporous sulfide, such as iron (Fe), cobalt (Co), nickel (Ni), or nanoporous carbides such as tungsten (W) or molybdenum (Mo) carbides.

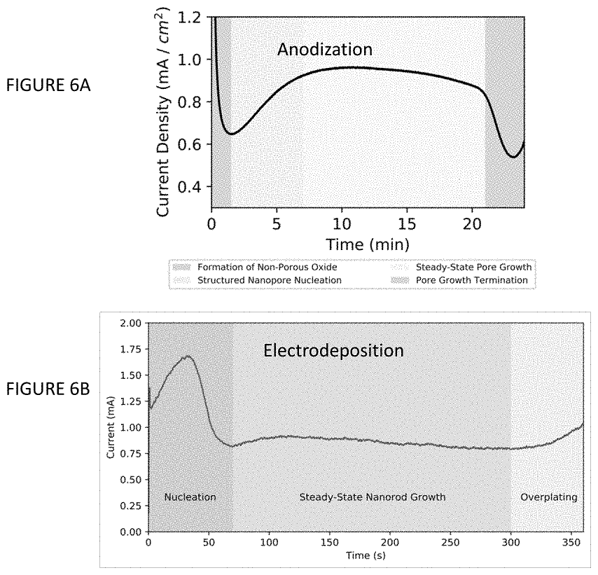

Referring now to FIG. 2B, this step involves anodizing the Al layer to completely convert it to nanoporous anodic aluminum oxide (AAO) that is subsequently used as a template for nanorod array fabrication. The anodization is carried out in an electrolytic bath under DC bias using a cathode with symmetry to the anode and is performed until all the Al metal is converted into Al₂O₃ and the pores in the layer penetrate through to the underlying conductive layer. The anodization can be performed as a function of time for a set period or by monitoring the current to determine when the Al metal has been completely converted to AAO. Such an anodization current versus time plot is shown in FIG. 6A. The electrochemical oxidation of Al metal can result in the “self-assembled” growth of a hexagonally ordered nanoporous Al₂O₃ matrix, in which the diameter and spacing of the nanopores can be controlled by varying the anodization voltage, the electrolytic chemical types, and the concentrations used. A uniform nanoporous AAO layer is formed by fully anodizing the Al in any of a variety of acidic electrolytes (e.g. sulfuric, oxalic, glycolic, phosphoric, malonic, tartaric, malic, citric or other acids) under DC voltage. A stainless-steel mesh cathode with symmetry to the anode (sample) is utilized. Nanoporous AAO can be formed with pore diameters ranging from 2-900 nm on a 35-980 nm pitch thus obtained by adjusting the process parameters of voltage, electrolyte type, electrolyte concentration, temperature, and surface pretreatments. Anodization is performed in the 20-200 V DC range depending on the desired AAO metrics. The pores can be widened and any remnant Al₂O₃ is cleared from the interface of the nanoporous AAO and the conductive layer using a post-anodization etch in 5% phosphoric acid at 38° C.

Referring to FIG. 2C, this represents the fabrication step where the template formation of metallic nanorod arrays within the nanoporous AAO layer is performed by the deposition of a plasmonic material into the AAO pores. The preferred method to form plasmonically active rods by template synthesis using a porous matrix templating technique is via electrochemical deposition of the plasmonic and/or catalytic material species into the openings of the porous matrix; although other methods such as chemical vapor deposition (CVD), pulsed vapor deposition (PVD), atomic layer deposition (ALD), and electroless chemical deposition can also be utilized as alternate approaches to rod formation. Plasmonic materials that can be used to form the nanorods include silver, gold, aluminum, copper, cobalt, chromium, iron, molybdenum, manganese, indium, nickel, palladium, platinum, rhodium, tantalum, titanium, titanium nitride, tungsten, silicon, tin, zirconium nitride, zinc, iridium, and others including various alloys, nitrides, and oxides of the aforementioned materials. Other plasmonic materials consist of highly doped semiconductors (Si, Ge, and III-V materials) and transparent conducting oxides. When formed by electro-deposition, the nanorods grow from the base upwards and can be made to completely or partially fill the AAO nanopores as desired by controlling the deposition rate and time. The plasmonically active rods can be used with the AAO layer retained (FIG. 2C) or removed (FIG. 2D) to expose the plasmonic rods to the local environment. The aluminum oxide can be removed by a chemical etch to expose the free-standing, vertically aligned plasmonically active rods. Alternatively, the porous matrix can be completely or partially removed by plasma or vapor etching techniques. The material embodiment with the AAO retained FIG. 2C has the qualities of being mechanically robust, chemically resilient, and radiation tolerant, with a large amount of the nanorod material protected by encasement in the AAO matrix. On the other hand, the exposed nanorod format of the invention has a highly enhanced surface area available for direct interaction with a variety of local environments. For example, a 1 cm² surface with plasmonic rods of 50 nm diameter, 500 nm length, and a density of 10¹⁰/cm² has 17 times the surface area compared to a planar material, and thus, 17 times the area for catalytic reactions to occur on. The present invention can be used to transduce optical energy into localized heating and the presence of high energy charge carriers and further transfer the thermal energy (heat) and charges to a gaseous or liquid material in contact with or close proximity to the plasmonically active rods.

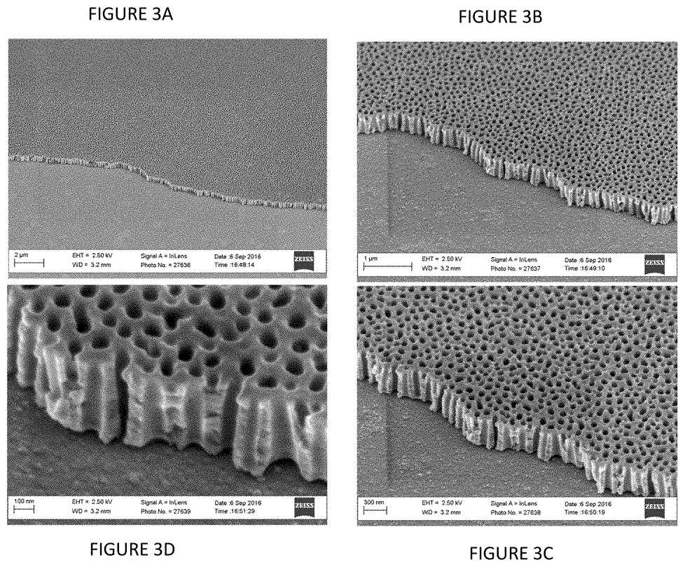

FIGS. 3A-3D show four scanning electron microscope (SEM) images of the present invention processed through step (B) in FIG. 2B and shows cross-sectional views of an AAO produced on silver coated glass at different magnifications to show both the long-range order and uniformity obtained in the plasmonic nanomaterial fabrication process. This series of SEM images taken at a 45° cross-sectional perspective show the silver layer on glass in the foreground with an ordered, uniform, and continuously produced AAO template layer in the back. This demonstrates the efficacy of the fabrication technique.

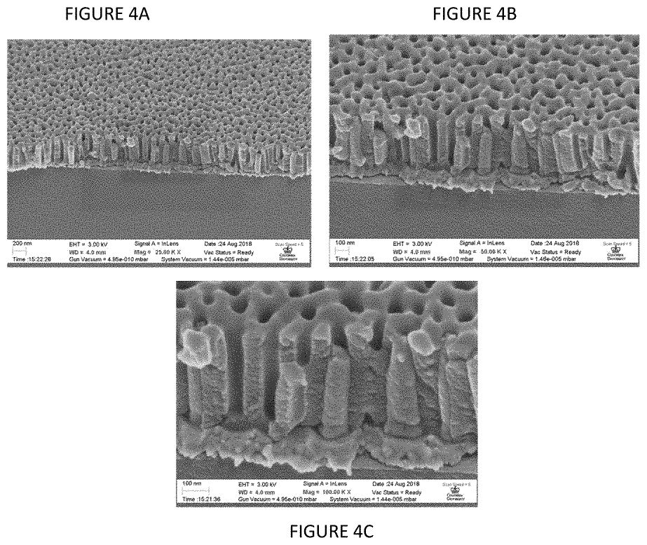

FIGS. 4A-4C show three SEM images of the present plasmonic nanomaterial invention processed through the stage shown schematically in FIG. 2C and shows 45° cross-sectional views of AAO produced on silver coated glass with nanorods embedded in the matrix. The images are shown at different magnifications to reveal the fine structure as well as the long-range order and uniformity obtained in the fabrication process. Nanorods of silver were formed via electrodeposition.

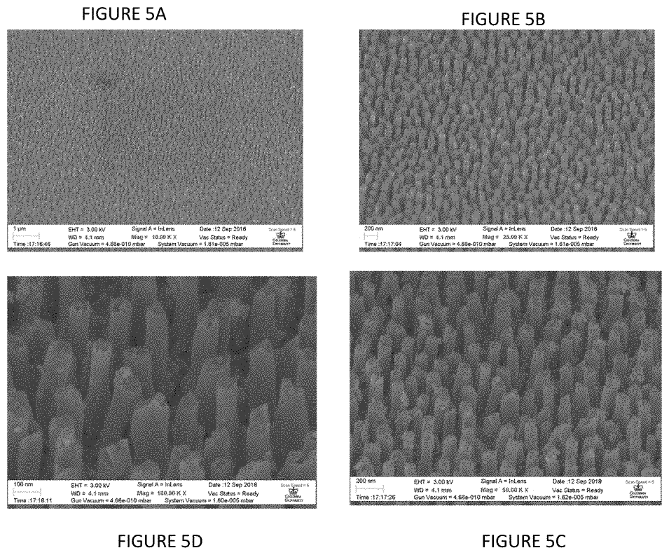

FIGS. 5A-5D show four SEM images of the present invention processed through the stage shown schematically in FIG. 2D and shows 45° cross-sectional views of exposed nanorods produced on silver coated glass. The AAO matrix has been removed via chemical etch using phosphoric acid. The plasmonic nanomaterial is presented at different magnifications to show the long-range order and uniformity obtained in the process. Nanorods are formed of via electrodeposition and comprise a high surface area format used in the photocatalytic flow reactor applications.

Referring to FIG. 6A in more detail, it shows a characteristic anodization current versus time plot used in fabricating an AAO template layer for the plasmonic nanorod array fabrication. FIG. 6A shows four regions of nanoporous oxide layer formation when performed in a 0.3 wt. % oxalic acid bath at 60 volts and 2° C. The various phases of oxide initiation, nanopore nucleation, growth, and termination at the seed layer interface, which are observed during anodization are shown in color code and can be used to automate the process for computer control by monitoring inflections in the current. In a similar regard, FIG. 6B is a characteristic electrodeposition current versus time plot from the electrodeposition of gold from a sulfite plating bath used to form gold nanorods in the AAO matrix. The trace is used to control and monitor the nanorod array fabrication and demonstrates how steady state growth can be used to adjust the nanorod length up to the point of overplating, prior to which the process must be terminated.

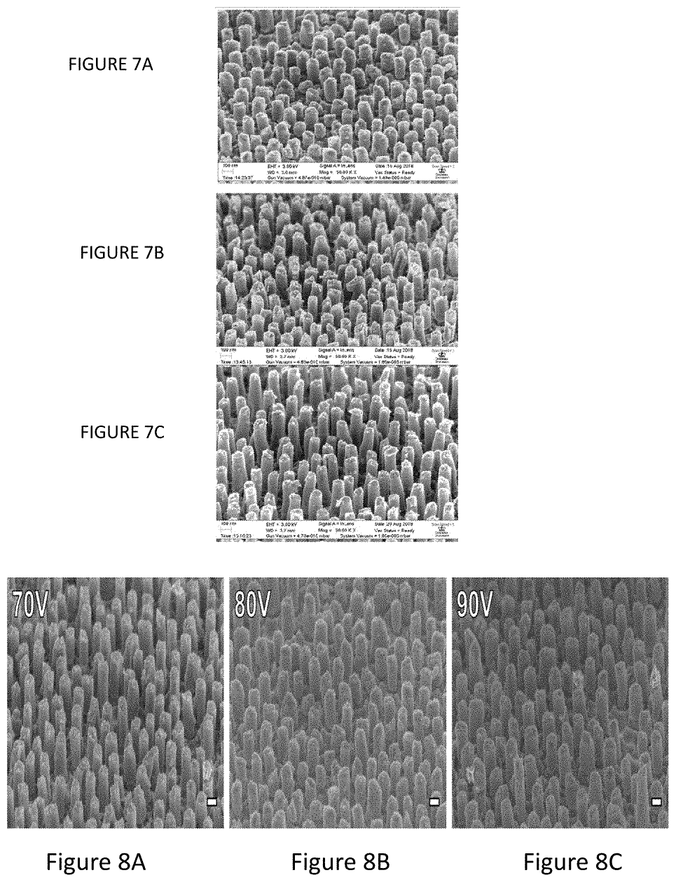

FIGS. 7A-7C show a series SEM images of plasmonic nanorod arrays with increasing lengths. FIG. 7A shows an SEM image of silver nanorods with an average length of 270 nm. FIG. 7B shows an SEM image of silver nanorods with an average length of 355 nm. FIG. 7C shows an SEM image of silver nanorods with an average length of 488 nm. Such variations are used to manipulate or tune the optical response of the material via shifts in the plasmon resonance wavelength which are shifted to longer wavelengths as the aspect ratio increases.

FIGS. 8A-8C show a series SEM images of plasmonic nanorod arrays with increasing spacing or pitch as controlled by adjusting the anodization voltage during AAO template formation. The nanorods were grown under 70V, 80V, and 90V constant anodization potentials in oxalic acid electrolyte. Pitch distances increased from 150 nm in FIG. 7A to 200 nm in FIG. 7C as a function of voltage. Such controllable variations are used to manipulate or tune the optical response of the material via shifts in the plasmon resonance wavelength.

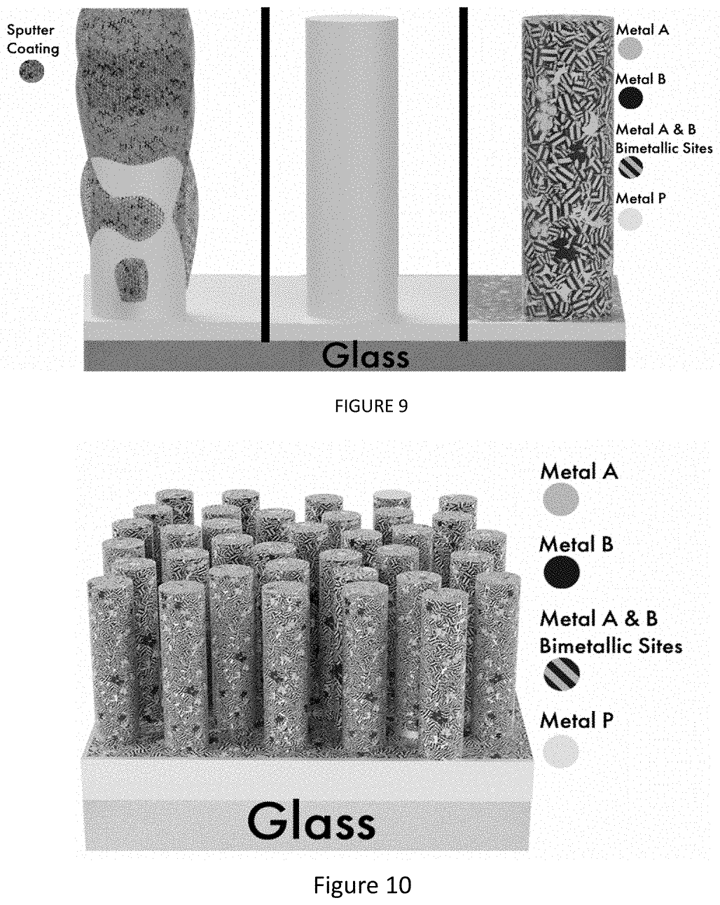

FIG. 9 is a schematic rendering representing before and after the catalytic activation of silver nanorods by the deposition of either a bimetallic surface coating (right), or a vapor deposited catalytic layer (left). There are numerous materials and techniques for catalytic activation including fabrication of the nanorods directly from the catalytic material. For example, Ag nanorod array samples can have a layer of Cu—Pd deposited on the surfaces either by physical vapor, or electroless deposition. It is anticipated that a large percentage of the nanostructures' silver surfaces will be covered with bimetallic layer (Cu—Pd for example) using electroless deposition as shown in the FIG. 9 right panel, while the type of coating obtained by physical vapor deposition is represented by the left panel schematic in FIG. 9. Electroless deposition can be significantly less expensive in a large-scale manufacturing scenario than vacuum vapor-based techniques and thus represents a preferred embodiment from an economic viewpoint. The co-electroless deposition of Cu and Pd is conducted at controlled rates and concentrations through the addition of Cu²⁺ and Pd²⁺ salts along with a suitable reducing agent to an ED bath. Beyond Cu—Pd, which are used for example, an ordinary practitioner skilled in the art will readily recognize that there exist numerous other metallic, alloy, and bimetallic layers that could be coated onto plasmonic nanorod arrays or that could be used in toto to fashion the nanorods from.

Again, referring to FIG. 9, the left panel shows the results of vapor deposition of a catalytic material or compound. For example, Cu and Pd can be co-deposited using dual sputtering sources in a vapor deposition system, where the power to the Cu and Pd targets would determine the relative percentage of each material in the subsequent bimetallic coating layer. Other approaches would include making the nanorods from Cu and then sputtering only a Pd layer. In this scenario, the nanorods can be made of silver, gold, aluminum, copper, cobalt, chromium, iron, molybdenum, manganese, indium, nickel, palladium, platinum, rhodium, tantalum, titanium, titanium nitride, tungsten, silicon, tin, zirconium nitride, zinc, iridium, and others including various alloys, nitrides, and oxides of these materials. These materials can be catalytic as is or can be coated or combined with other catalysts such as palladium, platinum, gold, ruthenium, rhodium, iridium, nickel, iron, chromium, zinc, or copper or such metal oxides. Other catalytic materials that can be deposited onto the nanorods include but are not limited to Cu/ZnO and Cu/ZnO₂, MnOₓ/m-Co₃O₄, In₂O₃/ZrO₂, and Pd—Zn alloys. Other methods of applying coatings include electrochemical deposition, chemical vapor deposition, thermal evaporation, electron beam evaporation, and atomic layer deposition.

Referring now to FIG. 10, the present catalytic plasmonic nanomaterial invention showing an array of metallic nanorods fabricated on glass with a bimetallic coating as may be obtained by electroless deposition. Cu and Pd bimetallic sites are observed in addition to monometallic islands of each metal and certain areas with no coating. Electroless deposition as used herein refers to using only one electrode and no external source of electric current as opposed to electrochemical deposition or plating cell, which consists of two electrodes, electroplating bath, and external source of current. However, the solution for electroless deposition needs to contain a reducing agent. A major benefit of this approach over electroplating, when scaling up and large-scale manufacturing is concerned, is that the power sources and plating baths are not needed, reducing the cost of production. ED has been used to produce controlled, bimetallic catalysts, and its combination with plasmonic nanorod arrays is a fundamental aspect of this invention.

A sputter coating can be applied using DC and RF sputtering and e-beam evaporation using a physical vapor deposition system to deposit metal thin-films and perform pre-sputter or pre-evaporation surface cleaning in situ. Under vacuum, Cu and Pd can be simultaneously co-deposited from separate 2″ targets using a rf magnetron for one and a dc magnetron for the other, to sputter deposit a bimetallic coating onto silver nanorod array samples as in FIG. 9. The deposition is performed at pressures of 3.5-8 mTorr, and power levels of 25-500 watts with the dc gun used for Pd and the rf on Cu operating at ˜40% less power than the dc source.

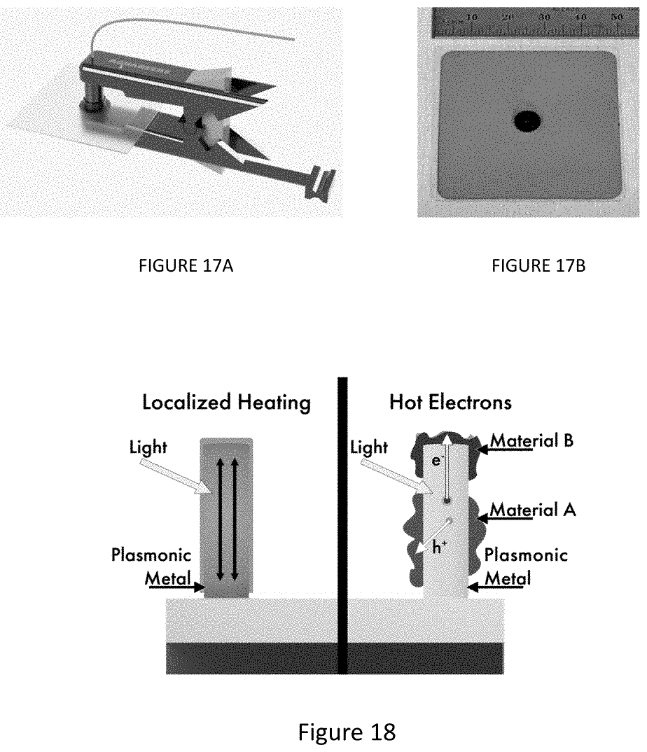

A custom-built electrochemical immersion process cell for batch processing of coated coupon samples is shown in FIG. 17A can be used to make electrical contact and carry the sample through the various baths. The cell operates using a spring-loaded design to clamp on the substrate and seal against the conductive surface with an O-ring fixture, ensuring a reliable water-tight isolation that prevents liquid infiltration leading to shorting. A single point electrode with a Pogo-Probe positioned in the center of the O-ring seal minimizes the contact area while maximizing the uniformity of the electrochemical processes. The electrical lead connects the sample to the outside electrical signal source as either the anode or the cathode to perform the anodization or electrodeposition processes, respectively. The body of the process cell is produced by 3D printing and can be readily made larger to accommodate differently sized or shaped substrates. The rigid body facilitates precise handling, orientation and easy electrical contact to the fragile glass substrate samples through the various process baths.

A fully processed 50 mm×50 mm plasmonic nanomaterial silver nanorod array sample, engineered on a WILLOW® Glass coupon, is presented in FIG. 17B showing the uniformity of the fabricated layer over the sample surface, aside from the unprocessed center spot where the O-ring seal for electrical contact is made. Reductions in the size of the center spot (7 mm diameter O-ring) are planned in future renditions of the process cell. WILLOW® glass is available in 300-meter-long rolls with widths of 1.3 meters, which sets an upper limit of the foreseeable substrate size.

The nanorods employed in this work had dimensions of ˜100 nm diameters and ˜500 nm lengths with center-to-center spacing of ˜200 nm, with some variations. More generally, the nanorods can have dimensions of about 50-150 nm diameters and about 400-2000 nm lengths with center-to-center spacing of about 75-300 nm. When illuminated at or near their “Plasmon Resonance” wavelength, the tiny nanorods are extremely effective optical antenna, absorbing over 90% of the incident radiation. When visible light is absorbed in the nanorod, the energy is converted into a plasmon—a collective electron oscillation that damps out on the order of a picosecond. The plasmon generates localized heat and hot carriers as shown schematically in FIG. 18, with both properties used to promote chemical reactions with adsorbates on the nanorods' surface. The localized nature of the plasmonic interaction effectively lowers the activation energy of reactants on the nanorod surface to undergo specific chemical reaction pathways.

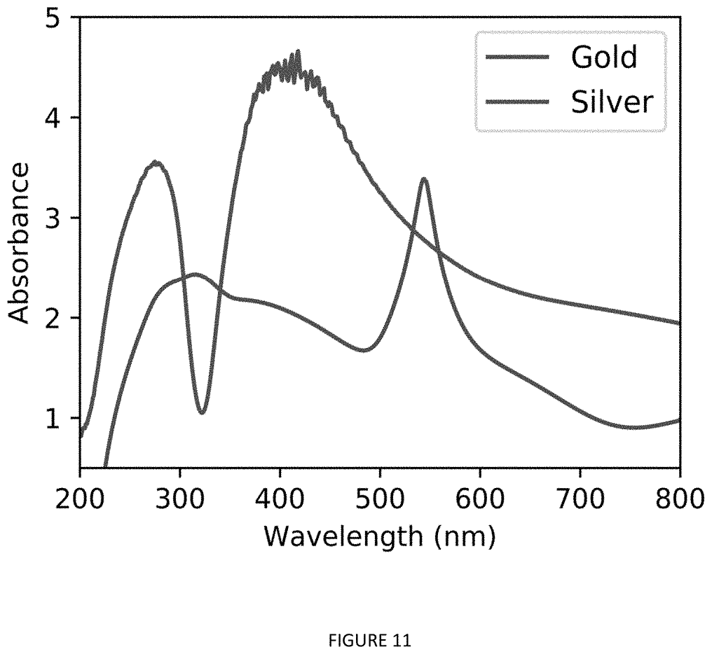

FIG. 11 shows absorbance versus wavelength data measured on gold and silver plasmonic nanorod array samples, showing the clear plasmon resonance peaks at 440 and 560 nm for the silver and gold respectively. Hot charge carriers that can be used to enhance catalytic reactions are excited at the resonance wavelength and off resonance at the wavelength corresponding to inter-band transitions, which can provide more energy for reactions.

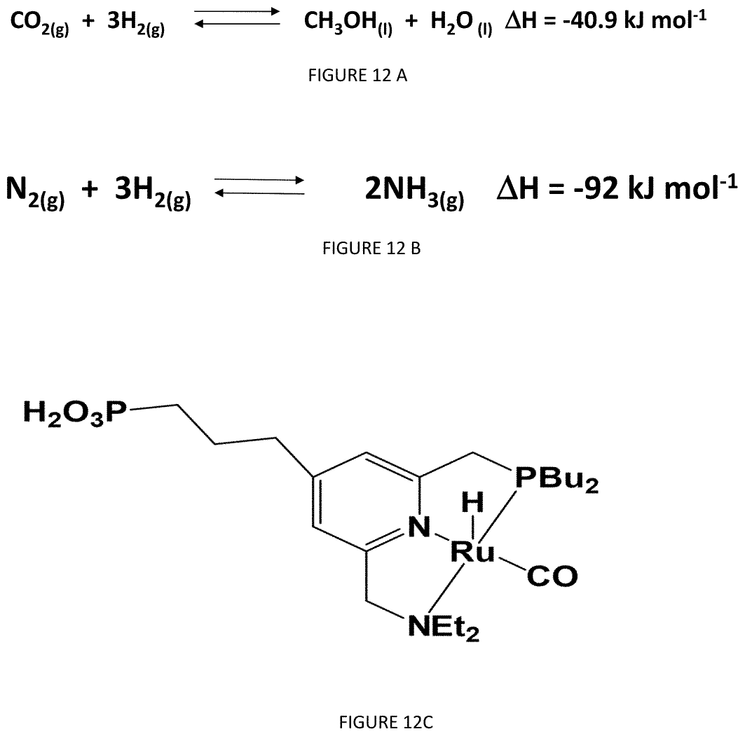

FIG. 12A shows chemical reaction and enthalpy change for the synthesis of methanol from carbon dioxide and hydrogen as is utilized in the catalytic optical flow reactor invention that operates by virtue of the catalytic plasmonic nanomaterial invention. Likewise, FIG. 12B shows the chemical equation and enthalpy change for the synthesis of ammonia from nitrogen and hydrogen as performed in the present invention. FIG. 12C shows one example of an organometallic homogeneous catalyst, functionalized with a phosphonic acid group, for its attachment either directly to silver nanorods having a native a nanometer-thick oxide layer, or to silver nanorods capped with a metal oxide such as, but not limited to Al₂O₃, CuO, Fe₂O₃, TiO₂, SnO₂, V₂O₅, WO₃, ZrO₂, and ZnO, in the present invention. It should be obvious to practitioners of ordinary skill in the art that other organometallic homogeneous catalysts could be linked to the silver nanorods using the phosphonic acid linker as described above. The present invention provides for the products made from the use of the flow reactor, such as methanol and ammonia.

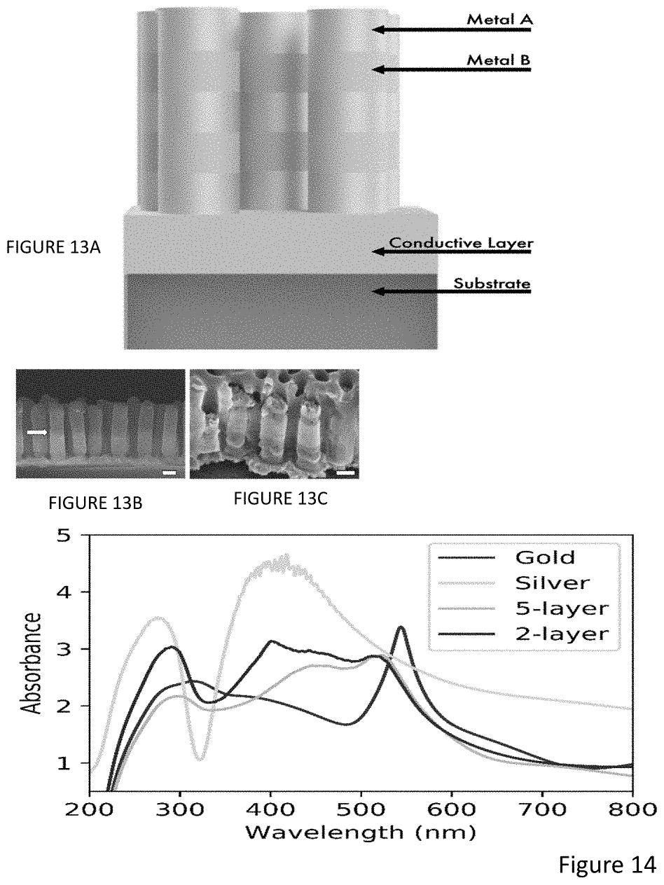

FIG. 13A is a schematic representation of a nanorod array with a layered substructure that is fabricated from alternating layers of two plasmonic materials such as gold and silver, or a plasmonic and catalytic material such as silver and nickel, or two catalytic materials such as copper and palladium. In this scenario, one layer can act as an antenna to absorb radiation, while the other is a more catalytically active “reactor” layer. Such a geometry is achieved by depositing various material layers by alternating between electroplating baths of the two or more desired metals or alloys. SEM images are shown in FIG. 11B of a free standing two-layer gold and silver nanorod array. SEM images of a five layered gold and silver nanorod array that is still embedded in the AAO template matrix is shown in FIG. 11C displayed with color enhancement on one nanorod used to clearly highlight and distinguish the layers. The layered nanomaterial can be used to tune the plasmonic response as shown in FIG. 14, which presents the absorbance versus wavelength data from FIG. 11 along with the uv-Vis absorbance spectra obtained from the two- and five-layer gold silver plasmonic arrays samples here. Features from both the gold and silver arrays are observed in the layered materials that can be used to manipulate the plasmonic spectral response to meet desired optical characteristics such as utilization of broad band solar power. Fully coated nanorods may hamper optical enhancements and the interaction of hot electrons with surface adsorbates vital to the catalytic process.

Nanorods with bimetallic nanocaps can also be produced by electrodepositing consecutive, nanometer-thick metallic layers of the bimetallic cap (such as Cu—Pd) onto the plasmonic metal nanorods (FIG. 2D). Annealing the material will result in mixing of the two metallic elements across all interfaces. This route allows control on the concentration of the two metals in the final bimetallic cap, which will be accomplished by controlling the relative thickness of the different layers in the alternating metallic layered structure.

Coupling effects occur within supported nanorod arrays and involve both rod-substrate and rod-rod coupling. In this strong coupling regime, the optical properties of the arrays are predominantly governed by inter-rod spacing, and the absorption efficiency is significantly enhanced by supporting the arrays on metal surfaces. This is important for the present invention since the plasmonic nanomaterial can be manufactured with great flexibility, using a variety of metals to form nanorods and a variety of metal or conducting surfaces below. In addition, it has been shown that the longitudinal mode of the plasmon can be tuned as a function of inter-rod spacing and aspect ratio. Most importantly, the coupling within unsupported and metal-supported arrays can redistribute the electric field to either the center or base of the nanorods, respectively, while propagating along the inter-rod axis, which is critical to performing catalytic reactions on the surfaces.

Localized surface plasmons excited on metal nanoparticles (e.g. gold, silver) decay non-radiatively into high energy hot electrons, with energies between the vacuum energy and the Fermi level. In this transient state, hot electrons can transfer into an H₂ molecule adsorbed on the nanoparticle surface, triggering facile dissociative reduction and consecutive reactions of the produced hydride and hydrogen atom with CO₂ or other molecules in the pathway of CO₂ reduction to methanol (CH₃OH) or even methane (CH₄). Such hot electrons are used to induce selective CO₂ conversion. Surface plasmons excited on metal nanoparticles (e.g. gold, silver) decay non-radiatively into high energy hot electrons and holes with energies between the vacuum energy and the Fermi level plus the absorbed photon energy. In this transient state, hot electrons can transfer into an H₂ molecule adsorbed on the nanoparticle surface, triggering facile dissociation and consecutive reactions of the hydride and the hydrogen atom with N₂ or other molecules in the pathway of N₂ reduction to ammonia (NH₃).

Catalytic nanoparticles usually refer to using nanoparticle dispersions as opposed to arrays of plasmonic nanorods being used in the current invention, which are referred to as heterogeneous catalysts with very high surface area, resulting in increased catalytic activity. A unique feature of the present invention, as compared to, for example, nanoparticle catalysts, is that separation of dispersions from reaction products is completely avoided with an insertable substrate carrier as used herein. Thus, the present catalytic nanomaterials in this invention can be inserted or removed from a reactor as one unit, separated from the reactor and from reaction products and recycled or serviced to replenish catalytic activity without resorting to sophisticated and costly separation techniques required for dispersions that can result in the loss of the catalyst altogether.

Alloys of two metals, called bimetallic, are used to create synergistic effects between the two metals in catalysis. For example, in the reduction of CO₂, one metal can have a stronger affinity to carbon, and the other to oxygen, making the C—O bond more susceptible to reduction. The catalytic plasmonic nanomaterial provides practically unlimited opportunities for creating such bimetallic nanocatalysts, either by creating nanorods from layers of different metals as in FIG. 13, or by coating or capping the nanorods with a bimetallic layer via vacuum deposition, utilizing electrodeposition, or by using electroless deposition to coat nanorods of one or more metals with nanometer-thick layers of other metals.

The catalytic plasmonic nanomaterial can be used in a large variety of reactions, such as the hydrogenolysis of C—Cl bonds in polychlorinated biphenyls, or in hydrogenation of halogenated aromatic amines, which is important in the synthesis of herbicides and pesticides as well as diesel fuel, and in hydrogenation of benzene to cyclohexane, and in hydrosilylation reactions.

Another group of synthetically important processes are organic redox reactions, and C [symbol] C bond formation (e.g., Heck coupling and Suzuki coupling reactions), where metals such as palladium have been used as catalysts, and where the catalytic plasmonic nanomaterial can enhance efficiency and lower cost by providing a facile and economical route for synthesis of highly expensive pharmaceuticals in small quantities, using clean energy, for immediate administration in developing countries using advanced flow reactors.

Heterogeneous catalysis and homogeneous catalysis are two main types of catalysis. In heterogeneous catalysis, the catalyst is in the solid phase with the reaction occurring on its surface. In homogeneous catalysis, the catalyst, a molecule—usually organometallic complex—is in the same phase as the reactants. Both processes have their benefits. For example, heterogeneous catalysts can, in principle, be readily separated from the reaction mixture, but reaction rates are restricted due to the limited surface area. However, while homogeneous catalysts can react very fast and provide a good conversion rate per catalyst molecule, they are miscible in the reaction medium, and it can be a painstaking and costly process to separate them from the reaction medium. This difficulty in removing homogenous catalysts from the reaction medium leads to problems in retaining the catalyst for reuse. The separation and recycling of catalysts are highly favorable since they are often very expensive. A possible solution for reusing homogeneous catalysts is their chemical attachment to a solid medium using a linker molecule as shown in FIG. 12C. There are huge numbers of homogeneous catalysts, and their utilization requires miscibility in the reaction mixture. In many cases, homogeneous catalysts contain very expensive rare metals, and when destroyed during workup, their use becomes an important cost factor, especially in large volume chemical reactions. Chemical attachment of homogeneous catalysts to the nanorods can be accomplished by modifying the organometallic catalytic complex with a functional group that has a strong affinity to the nanorod surface.

The use of homogeneous catalysts requires design of the solvent system. In many cases, a liquid-liquid biphasic catalytic system is used, which consists of a catalyst phase containing the dissolved catalyst and a product phase. Usually, water, alcohols, ionic liquids, fluorocarbons, supercritical fluids, and gas expanded liquids have been used as the catalyst phase. In such a biphasic system, the catalytic reaction occurs at the interface of the two phases, or phase transfer agents may be added to facilitate the reaction.

The catalytic plasmonic nanomaterial can serves as a bridge between heterogeneous and homogeneous catalysts, providing the benefit of maintaining high reaction rates. Different from well-dispersed functionalized nanoparticulate catalysts, the catalytic plasmonic nanomaterial can simply be removed from the reactor, rejuvenated, and reused.

Using different surface attachment chemistries, plasmonic nanostructures permit multiple catalytic functionalities on the same plasmonic nanomaterial, hence providing a unique system for performing a cascade of catalytic reactions, where the product of one catalytic reaction can further react at neighboring catalytic site on the same nanorod, or on adjacent nanorods, etc.

One particularly important and useful catalyst support is magnetic nanoparticles. Such nanoparticles enable immobilization and magnetic recovery of the catalyst in the presence of a magnetic field, and its reuse. The present invention is superior to magnetic nanoparticles, providing immediate and facile catalyst separation and reuse, without the need of magnetic force or tedious extra steps.

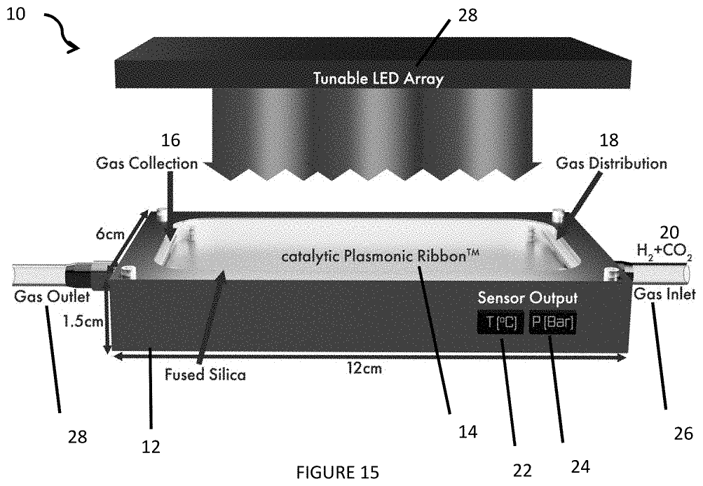

Regarding the present invention, the catalytic plasmonic nanomaterial can be used in an optical flow reactor 10, such as one represented in FIG. 15, such as for synthesis of methanol, ethylene, ammonia, or other products. Flow reactors provide a uniquely powerful use of the catalytic plasmonic nanomaterial invention. While traditional thermal batch reactors and processing techniques have been proven over decades of use, they are very costly due to high temperatures and pressures used and have efficiency, quality control, and safety shortcomings that could be particularly troublesome in specialty chemical production. Flow reactors promote a chemical reaction in a continuously flowing stream of reactants that flow over the illuminated plasmonic catalyst. Pumps move or flow input gases or fluids into a defined volume (chamber 12) containing the catalytic plasmonic nanomaterial 14, where mixing is achieved through a gas distribution manifold 18 and then products and unused gases or fluids are collected through a gas collection manifold 16. The dual manifold system delivers and collects gas uniformly to reduce stagnant, or dead zones in the reactor and gives high control over the flow rates. In the flow reactor 10, reactive components 20 are pumped together at a mixing junction inside the gas distribution manifold 18 and flowed down the irradiated chamber 12 containing the catalytic plasmonic ribbon 14. The chamber 12 can be stainless steel or aluminum for example and has an O-ring seal between the base fixture and the hermetically sealed fused silica (or quartz) window upper fixture allowing it to operate at pressures up to 12 Bar. Fused silica will allow the full UV irradiation across the plasmonic spectrum of the nanorods to be utilized in promoting the catalytic reaction. An output gas passes through a sample loop with direct injection into a gas chromatograph (GC) with thermal conductivity detector to determine the constituents. The reactor 10 has mass-flow controllers for gas feed and thin film heater elements not shown.

An LED array 18 is utilized for illumination including wavelength specific units such as UV SMD LEDs from Boston Electronics and CXA Chip on Board (COB) LED arrays, or broadband visible emitters produced by CREE to achieve an LED spectrum from 250 nm to 1000 nm with a spectral output of ˜800-1000 watts/m². An alternate light source is a 150 W Xenon Lamp providing a spectrum of 200-1000 nm that can be selectively narrowed using optical filters as necessary. The LED array 28 can be positioned over the chamber 12 to effectively illuminate the catalytic plasmonic nanomaterial 14. Thermocouple temperature sensors 22 and a gas-liquid pressure sensor 24 are incorporated into the chamber 12 for reaction condition monitoring and data logging. Constant temperature control can be provided to the chemical reaction chamber. Preferably, the LED wavelength matches a plasmon resonance wavelength of the catalytic plasmonic nanomaterial.

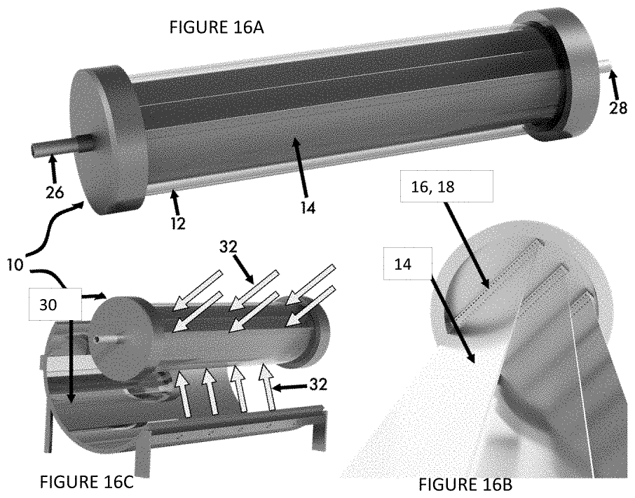

Referring now to FIGS. 16A-16C in detail, they show representations at various perspectives of how the catalytic plasmonic nanomaterial invention can be field deployed in a tubular flow reactor mounted in a concentrating solar collector to provide optical power for the photocatalytic synthesis of different chemicals and compounds using solar energy. FIG. 16A shows a schematic representation of a tubular flow reactor 10 with gas input 26 and outlet 28 flanges that cap a transparent reactor tube (chamber) 12 that houses the catalytic plasmonic nanomaterial 14. FIG. 16B shows a detailed representation inside the tubular flow reactor 10 with details of the gas distribution/collection manifold 16/18 that ensures uniform distribution of well mixed reactants over the catalytic plasmonic nanomaterial 14. The FIG. 16C schematic shows a field deployed scenario for the tubular flow reactor 10 that is coupled with a solar concentrating mirror 30 to provide optical power 32 from its surface. Optical energy 32 is also incident directly onto the reactor surface. The flow chemistry allows only small amounts of hazardous intermediates to be formed at any time in the reaction chamber. This provides safety benefits as the reactor operates under steady-state conditions. Constant temperature control can be provided to the chemical reaction chamber. Preferably, the LED wavelength matches a plasmon resonance wavelength of the catalytic plasmonic nanomaterial.

Using the flow reactor 10, fuels and chemicals including oxygenates like methanol, hydrocarbons like ethylene, or non-carbon compounds like ammonia can be synthesized. The present invention provides for a method of photocatalytic synthesis of methanol, by using the catalytic plasmonic nanomaterial to convert CO₂ and H₂ to methanol using optical power. More specifically, the method includes photo-absorbing to activate the nanomaterial, generating heat and energetic charge carriers from the activated nanomaterial, thereby driving catalytic reaction between catalyst deposited on nanomaterial and ambient reactants, and producing methanol. Reactants input into a reactor to produce the methanol and ammonia can be CO₂, H₂O, or CH₄. Example 3 further describes the use of the flow reactor 10.

Most generally, the present invention provides for a method of synthesizing useful chemicals from greenhouse gases such as CO₂ and CH₄ and waste gases that are used as sources in the synthesis of chemicals and fuels by using catalytic plasmonic nanomaterial to convert chemicals from greenhouse gases to useful chemicals (i.e. methanol and ammonia) using optical power.

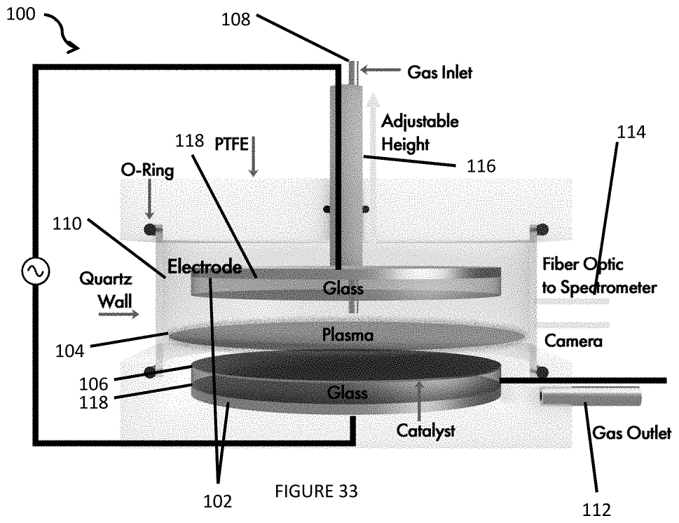

Referring now to FIG. 33, a hybrid plasma-plasmonic reactor 100 is presented employing the catalytic plasmonic nanomaterials. This dielectric barrier discharge (DBD) type system creates a non-thermal plasma. Plasma is one of the four fundamental states of matter. It consists of a gas of ions—atoms which have some of their electrons removed—and free high energy electrons. In such non-thermal plasmas, the gas phase is far from equilibrium, and the system is complex, requiring a step evolution: first, radicals are formed by electron impact reactions, and then, propagation and recombination reactions occur and result in the final products. In a non-thermal plasma, the bulk gas temperature can be T_{G}=300-500 K, while electron temperatures range between Tₑ 10⁴-10⁵ K (˜1-10 eV). The ‘hot’ electrons generated in the plasma zone offer a convenient way to carry out reactions that otherwise require high temperatures and pressures to produce added-value chemicals, for example the reaction of CO₂ and CH₄, to produce syngas (CO₂+CH₄→2H₂+2CO), in the “Dry Reforming of Methane” (DRM) process, which can be further processed into Fischer-Tropsch liquids or methanol.

Still referring to FIG. 46, disc electrodes 102 ˜5 cm in diameter with an adjustable gap 116 (1-5 mm) include a layer of the catalytic plasmonic nanomaterials 118 ignite a plasma 104 from gas under test in a PTFE and quartz constructed reactor 100. The reactor 100 is designed to accommodate pressures from 0.1 to 5 Bar and is operated near atmosphere with no external heating or cooling supplied. Bias can be applied to a catalyst layer 106 to manipulate the interaction with the plasma discharge zone. The spacing between the disc electrodes 102 varies mechanically, while a bias on the catalytic layer 106 allows plasma manipulation. Gas enters at a gas inlet 108 into a reaction chamber 110. Gas or liquid products can be collected from the base or sent directly to an inline gas chromatograph (GC) at a gas outlet 112. The system is equipped with optical spectrometer by a fiber optic 114 and 4k resolution camera to monitor the environment in real time, pressure sensor and thermocouples, and voltage and current sensors (not shown). Power is supplied through a high-voltage (1-12 kV) supply coupled to a function generator acting in pulse or direct mode from 50 Hz-30 KHz, and the signal can be synched to the sample bias.

Therefore, the present invention provides for a plasma reactor device 100 that utilizes plasmonic catalytic nanorod arrays for synthesis of fuels and chemicals including methanol or ammonia, including a reaction chamber 110 containing a first adjustable disc electrode 102 having first catalytic plasmonic nanomaterial layer 118 thereon and a second adjustable disc electrode 102 having a second catalytic plasmonic nanomaterial layer 118 thereon, the reaction chamber 110 including a gas inlet 108 for flowing gas containing reactive components over the first and second catalytic plasmonic nanomaterials 118 and a gas outlet 112 for collecting synthesized gas products, wherein the first and second catalytic plasmonic nanomaterial layers 118 ignite a plasma 104 from gas introduced into the reaction chamber 110 and synthesize fuels and chemicals.

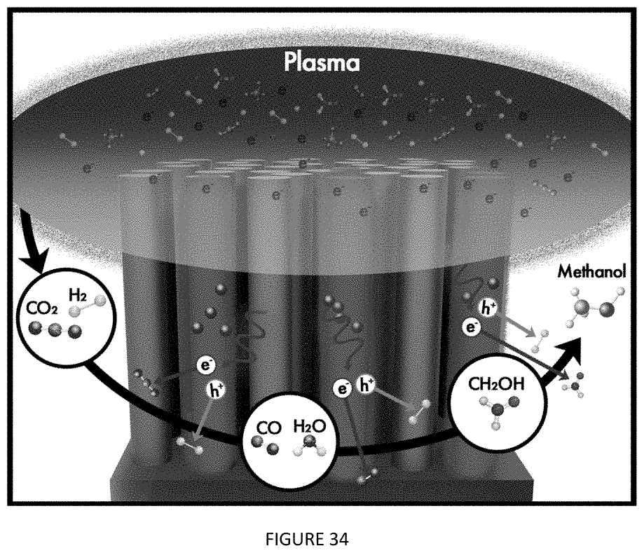

The coupling and reaction mechanisms present in the reactor 100 are presented in FIG. 34, schematically showing some of the phenomena and reaction pathways that can occur. However, due to the numerous variables and related chemical pathways only a partial display of some relevant features can be shown schematically. The various sub constituents of CO₂ and CH₄, such as CO, O, H₂, etc. are shown, which adsorb onto the catalytic nanorod surface and undergo chemical transformation. A myriad of effects are at play to lower the activation energies, including hot carriers, localized heat (vibrational energy), optical excitations, and direct ionization and dissociation In addition to the targeted synthesis on methanol (CH₃OH), other oxygenates like acetic acid (CH₃COOH), formaldehyde (HCHO), and ethanol (C₂H₅OH) can be formed as well as hydrocarbons like acetylene (C₂H₂), ethylene (C₂H₄), and ethane (C₂H₆).

Direct synthesis pathways have been demonstrated using plasma catalytic reactors to produce hydrocarbons, and oxygenates such as methanol, ethanol, formaldehyde, ether, acetic acid, and formic acid, and also ammonia, under conditions that require 2-3 times less energy than indirect syngas techniques.

Photons from the plasma can stimulate plasmons in catalytic plasmonic nanomaterials, with associated hot electrons and localized heating used to chemically alter adsorbates. The use of plasmonic materials within the plasma environment provides a means to control plasma energy on the nanoscale through synchronistic coupling of low electron density plasma excitations (10¹²/cm³) into high electron density (10²²/cm³) metallic nanostructures.

In a plasma-plasmonic reactor, neutral, high-energy species in the plasma can get very significant stabilization by coordinating with catalytic transition metal surfaces. ‘Hot’ electrons from the plasma excitations in the plasmonic nanostructures can further accelerate reaction kinetics, providing a parallel reaction channel and increased product yield.

Therefore, the present invention provides for a method of producing chemicals by stimulating plasmons in catalytic plasmonic nanomaterials with photons in a plasma catalytic reactor and producing chemicals. A non-thermal plasma is created between the two-disc electrodes 102 that can interact with the plasmonic catalytic nanomaterial. Optical excitation from the plasma in the plasmonic structures work in concert with numerous species of excited molecules, atoms, ions, electrons, radicals, and photons in the plasma to break the chemical bonds of stable molecule like CO₂, CH₄, H₂O and others and interact with the catalytic surfaces for the direct synthesis of higher order compounds including oxygenates like methanol, hydrocarbons like ethylene, or compounds like ammonia; The use of plasmonic materials within the plasma environment provides a means to control plasma energy on the nanoscale through synchronistic coupling of low electron density plasma excitations (˜10¹²/cm³) into high electron density (˜10²²/cm³) metallic nanostructures.

The catalytic plasmonic nanomaterials of the present invention provide several advantages. Traditional methods for thermally activated catalytic synthesis of chemicals and fuels are energy intensive, inefficient, and have a massive carbon footprint. Plasmonic nanomaterials can facilitate advanced photocatalytic processes and reactor designs. Plasmonic nanostructures made from, or coated with, catalytic materials provide a localized means for transducing optical energy into heat and energetic charges that can effectively lower the activation energies for chemical synthesis compared to thermal techniques. The accumulation of greenhouse gases (carbon dioxide, methane, etc.) in the atmosphere from fossil fuels usage leads to global warming and climate change. These carbon-based waste gases can be utilized as source materials to synthesize the carbon-based chemicals and fuels traditionally derived from fossil sources that are required for global infrastructure and economies. This can be achieved in a carbon neutral or negative manner through plasmonically enhanced photocatalytic reactions driven by solar energy. Plasmonic catalytic materials for photocatalysis in the prior art typically consist of nanoparticle dispersions that are hard to handle, and difficult to properly place, recover, and rejuvenate. The present invention allows for the attachment of plasmonic nanostructured (rod) arrays to optically compatible surfaces that can be readily positioned in a photocatalytic reactor, and manipulated, removed, recycled, or reactivated as necessary.

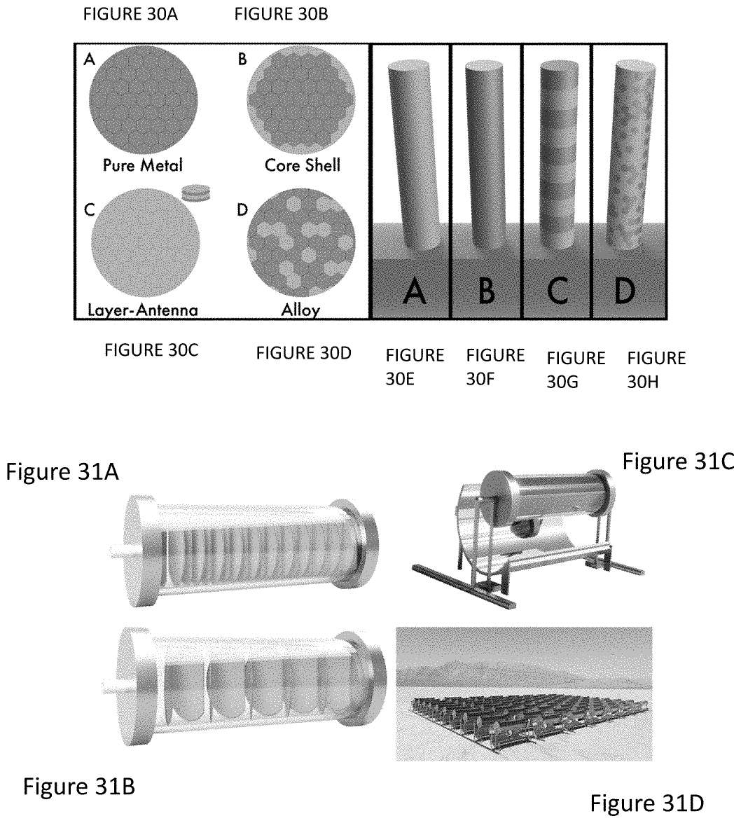

Referring to FIGS. 30A-30H, a variety of nanorod morphologies are shown in top down planar and cross-sectional views that can be exploited in the fabrication process of the current nanomaterial invention to optimize the performance of catalytic and plasmonic materials fabricated into nanorod arrays. FIGS. 30A and 30E show a pure metal nanorod, FIGS. 30B and 30F show the core-shell structure, FIGS. 30C and 30G show the layered structure, and FIGS. 30D and 30H show an alloy or bimetallic nanorod.

Currently, the illumination of plasmonic photocatalytic materials in dispersion or packed-bed geometries is inefficient and non-uniform consisting of hot spots with large dead-zones in between. The plasmonic photocatalytic structures of the present invention can be mounted on an optical material such as glass that allows light transparency and can also be used as a light guide to stimulate the plasmonic response internally. Precise control over the dimensions, structural morphology, and chemical constituents realized in the production of plasmonic photocatalytic materials currently is limited, inexact, and difficult to manipulate. Using an aluminum oxide template technique in the present invention to electrochemically form arrays of vertically aligned nanorods gives precise control over their dimensions and material constitution over a wide range of parameters. Pure materials, alloys, layers (antenna-reactor) and core-shell structures (by fully coating the nanorods) are readily manufactured by electrodeposition. Active coatings, processes, and surface treatments are applied to exposed nanorods after oxide removal utilizing chemical or physical techniques. Performing multistep electrochemical processes for nanofabrication requires a robust and rinseable surface electrical connection that is isolated from the chemical baths yet fully immersible in caustic chemical solutions. In the present invention, an electrochemical immersion cell can be used with a spring-loaded polymer design to mount and seal against a conductive surface (FIG. 17A). The mechanism uses a pogo-probe isolated through an O-ring seal for electrical connection to minimize contact area while providing uniform process results.

Currently, the design of photocatalytic reactors is limited by the catalytic platform used and most are not amenable to direct solar power. Plasmonic photocatalysts mounted on glass or other optical substrate material (which can be flexible) can be used to maximize possible design versatility and light utilization. The optical substrate can be in a planar, fiber, or tube format and can be used to transmit or guide optical power to the photocatalytic nanostructures. Reactors can utilize solar irradiation directly or be fed light externally. Currently, plasma powered catalysis shows promise for lowering the activation energies for important chemical and fuel synthesis reactions but designs that synergistically couple photocatalytic materials into plasma reactors efficiently and controllably are lacking. Plasmonic photocatalysts mounted on dielectric substrates in the present invention can be accurately positioned in multiple reactor designs that employ both the substrate and nanomaterials actively and synergistically both to generate a plasma and utilize its physical manifestation by absorbing optical energy and promoting surface reactions with chemicals that have either been altered or energetically excited by the plasma. By example the input gas can be a mixture of carbon dioxide and methane from which the plasma is initiated, but the reaction can yield higher hydrocarbons, hydrogen, synthesis gas, alkanes, alcohols, carboxylic acids, alkenes, or aromatics.

This application incorporates by reference the following documents: U.S. patent application Ser. No. 15/810,341 filed on Nov. 10, 2017, U.S. Provisional Patent Application No. 62/544,093, filed on Aug. 11, 2017, U.S. Provisional Patent Application No. 62/420,759, filed on Nov. 11, 2016, U.S. patent application Ser. No. 13/016,845 filed on Jan. 28, 2011, U.S. patent application Ser. No. 12/759,537 filed on Apr. 13, 2010, U.S. patent application Ser. No. 12/281,511 filed on Sep. 3, 2008, U.S. patent application Ser. No. 12/185,773 filed on Aug. 4, 2008, U.S. patent application Ser. No. 11/917,505 filed on Jul. 16, 2008, U.S. patent application Ser. No. 12/166,715 filed on Jul. 2, 2008, U.S. Provisional Patent Application No. 61/060,011 filed on Jun. 9, 2008, U.S. Provisional Patent Application No. 60/946,821 filed on Jun. 28, 2007, and U.S. Pat. No. 7,713,849 filed on Aug. 18, 2005, all of which are incorporated by reference in their entireties for all that they teach.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLE 1

### Fabrication and Characterization of Plasmonic Nanomaterials

Silver plasmonic nanomaterial samples are prepared on 50 mm×50 mm coupons of CORNING® WILLOW® Glass. The coupons are first coated with a 10-30 nm Ti adhesion layer, followed by a 20-50 nm Ag layer and then an Al layer of 200-800 nm thick. The deposition is performed via sputter and/or e-beam evaporation under ultra-high vacuum. The coated coupons are then mounted in an electrochemical immersion process cell which is used to make electrical contact using a pogo-probe and O-ring assembly and carry the sample through the anodization and electroplating steps of the nanofabrication process. The pogo probe allows ease of handling as the samples are electrochemically processed with minimal contact area.

The plasmonic nanomaterial is fabricated on coated glass by first completely anodizing the Al layer and forming a nanoporous AAO template used for nanorod array synthesis. It is well established that the electrochemical oxidation of Al metal can result in the “self-assembled” growth of a hexagonally ordered nanoporous Al₂O₃ matrix, in which the diameter and spacing of the pores can be controlled by varying the anodization voltage, and the electrolytic bath. A uniform AAO layer is formed by fully anodizing the Al in a variety of acids (e.g. sulfuric, oxalic, glycolic, phosphoric, malonic, tartaric, malic, or citric acids) under DC voltage. A stainless-steel mesh cathode 2:1 aligned parallel to the anode (sample) at 12″ spacing is utilized. The nanoporous AAO can be formed with pore diameters ranging from 2-900 nm on a 35-980 nm pitch range by adjusting the process parameters. Anodization is performed in the 20-200 V DC range depending on the desired AAO metrics. In a representative process, the Al layer is anodized in a 0.3 wt % oxalic acid bath at 5 degrees C. and 90 volts until all the Al metal is consumed (10-12 minutes) and the pores in the Al₂O₃ penetrate through to the conducting Ag layer. The pores are subsequently widened and remnant Al₂O₃ cleared from the bases at the AAO-Ag interface by etching in a 5 wt % solution of phosphoric acid at 38 degrees C. The AAO layer is optically transparent at this point, where the penetration of the pores through to the underlying Ag layer can be readily observed in this cross-sectional view (FIG. 3A-3D). Pore thru-penetration is required for subsequent robust mechanical and electrical contact of the nanorods to the underlying conducting layer.

Silver is electrodeposited from a pH of 10.0 solution of silver succinimide at 48° C. Silver nanorod arrays will be produced by standard dc electrolysis conducted at 0.7 Volts that yields a nanorod growth rate of ˜200 nm/minute. After plating, the AAO matrix will be fully removed by etching using a 5 wt % solution of phosphoric acid at 38° C. or sodium hydroxide at 30° C.

A variety of nanorod array metrics can be fabricated to determine the optimal configuration and plasmonic spectra for this catalytic application. As previously mentioned, the bandwidth (wavelengths) of the plasmonic response in the material can be tuned by adjusting the geometry (diameter, length, or pitch) of the nanorods or the thickness of the silver underlayer. As the nanorod length (aspect ratio) is increased, the primary transverse resonance mode decreases in magnitude as a longitudinal mode emerges and begins dominating at ever-longer wavelengths. While the resonance redshifts from 350 nm to 850 nm as the aspect ratio increases from 1 to 8; as the Ag thin-film layer thickness is increased, the resonance wavelength then blue shifts. Thus, there are numerous degrees of freedom in the catalytic plasmonic nanomaterial system that can be exploited to engineer the optical response for the desired application. For example, SEM images of silver nanorod arrays engineered with a range of lengths from 270-488 nm are presented in FIGS. 7A-7C.

### EXAMPLE 2

### Catalytic Activation of Silver Plasmonic Nanorod Arrays

Cu—Pd bimetallic catalysts have greater CH₃OH formation rates than either Cu or Pd monometallic catalysts exhibit and are effective at promoting the reaction CO₂+2H₂→CH₃OH. The preparation of a stoichiometrically controlled bimetallic layer on the nanorod surfaces can be performed with varying the coverage of the Cu—Pd bimetal. By controlling the surface stoichiometry of Cu and Pd on the Ag nanostructures, optimal formulations of bifunctional, bimetallic catalysts can be prepared. The Pd sites provide active locations for the dissociative adsorption of H₂, while the adjacent, or vicinal Cu sites, promote dissociative adsorption of CO₂ to form adsorbed CO and oxygen species, which subsequently undergo facile reduction to form CH₃OH and H₂O as products. In the present invention, a bimetallic layer is applied to the silver nanorod arrays to catalytically activate the plasmonic nanomaterial. Such coated nanorods are represented in FIG. 9 and FIG. 10.

Electroless deposition can provide a more uniform coating with higher-percentage coverage than other methods such as sputtering, since the surface of the plasmonic nanomaterial is highly non-planar and contains areas that may be inaccessible for line-of-sight methods such as physical vapor deposition techniques. Electroless deposition is conducted using aqueous solutions at ambient or near ambient conditions of temperature and pressure and is commonly used with porous materials. Electroless deposition is a catalytic or autocatalytic process whereby a chemical reducing agent reduces a metallic salt or salts onto specific sites of a pre-existing catalytic surface. In this case the catalyst surface is the nanostructured Ag surface and the support is the flexible glass substrate. The co-electroless deposition is conducted at controlled rates and concentrations through the addition of Cu²⁺ and Pd²⁺ salts along with a suitable reducing agent to comprise the electroless deposition bath. The Cu—PD is performed through the continuous electroless deposition of a reducing agent such as hydrazine or formaldehyde and two metal salts (Cu²⁺ and PdCl₄²⁻) into a stirred aqueous bath containing the Ag plasmonic nanomaterial. The extents and rates of Cu and Pd deposition are determined by analysis of the Cu²⁺ and PdCl₄²⁻ salts remaining in solution as a function of deposition time. The extent and rates of Cu and Pd deposition is utilized to optimize the Cu²⁺ and PdCl₄²⁻ salts remaining in solution as a function of deposition time. The extent of deposition of the bimetallic layer can vary from sub-monolayer, “island” type coverage to multiple monolayers covering the entire surface. This targeted deposition will result in the formation of only bimetallic surfaces of Cu and Pd sites, and not a wide range of compositions.

The nanorods can also have nanometer-thick metallic islands, which are prepared by electroless deposition, thus providing a large catalytic surface area, and where the solvent between nanorods is expected to have a higher and more stable temperature than that in the bulk solvent above. The preparation of electroless deposited metal catalyst allows the preparation of many catalytic plasmonic nanomaterials using single metals and metal alloys prepared using this method.

### EXAMPLE 3

### Photocatalytic Synthesis of CH₃OH from CO₂ and H₂ Using Optical Flow Reactor

An optical flow reactor design that utilized for the light induced synthesis of methanol from CO₂ and H₂ via the present catalytic plasmonic nanomaterial invention is shown in FIG. 15. This is a continuous gas-phase flow-reactor design incorporating feed and product sampling and analysis. The volume of the reaction vessel volume is 50 mL and it accommodates two 50×50 mm square coupons of the catalytic plasmonic nanomaterial. It has a low-profile bed for short residence time short, i.e. 30 seconds at 100 sccm flow (standard cubic centimeters per minute). The system is mounted in a heating mantle to be used as needed, although optical power from the LED array is the primary driver in the reaction.

Regarding the present invention, the catalytic plasmonic nanomaterial can be used in a flow reactor 10, such as one represented in FIG. 15, such as for synthesis of methanol, ammonia, or other products. Flow reactors provide a uniquely powerful use of the catalytic plasmonic nanomaterial invention. While traditional batch reactors and processing techniques have been proven over decades of use, they have energy consumption, carbon footprint, efficiency, quality control and safety shortcomings that could be particularly troublesome in specialty chemical production. Flow reactors promote a chemical reaction in a continuously flowing stream of reactants that flow over the illuminated plasmonic catalyst. Pumps move or flow input gases or fluids into a defined volume (chamber 12) containing the catalytic plasmonic nanomaterial 14, where mixing is achieved within seconds through gas distribution manifold 18 and then product and unused gases or fluids are collected through a gas collection manifold 16 that deliver and collect gas uniformly and reduce stagnant, or dead zones in the reactor. In the flow reactor 10, reactive components 20 are pumped together at a mixing junction inside a distribution manifold 18 and flowed down the irradiated chamber 12 containing the catalytic plasmonic nanomaterial 14. The chamber 12 is stainless steel with O-ring seal and has a hermetically sealed fused silica (or quartz) window to operate at pressures up to 12 Bar. Fused silica will allow the full UV irradiation across the plasmonic spectrum of the silver nanorods to be utilized in promoting the catalytic reaction. An output gas passes through a sample loop with direct injection into a gas chromatograph (GC) with thermal conductivity detector to determine the constituents. The reactor 10 has mass-flow controllers for gas feed and thin film heater elements not shown.

An LED array 28 is utilized for illumination including UV SMD LEDs from Boston Electronics and CXA Chip on Board (COB) LED arrays or visible emitters produced by CREE, to achieve an LED spectrum from 250 nm to 1000 nm with a spectral output of ˜800-1000 watts/m². An alternate light source is a 150 W Xenon Lamp providing a spectrum of 200-1000 nm that can be selectively narrowed using optical filters as necessary. The LED array 28 can be positioned over the chamber 12 to effectively illuminate the catalytic plasmonic nanomaterial 14. Thermocouple temperature sensors 20 and a gas-liquid pressure sensor 22 are incorporated into the chamber 12 for reaction condition monitoring and data logging.

The present invention provides for a method of photocatalytic synthesis of methanol, by using the catalytic plasmonic nanomaterial to convert CO₂ and H₂ to methanol using optical power. More specifically, the method includes photo-absorbing to activate the nanomaterial, generating heat and energetic charge carriers from the activated nanomaterial, thereby driving catalytic reaction between catalyst deposited on nanomaterial and ambient reactants, and producing methanol.

Flow reactors 10 utilizing the catalytic plasmonic nanomaterial 14 can be pressurized, allowing reaction of gaseous starting materials 20 (CO₂ and H₂ in the production of methanol, or N₂ and H₂ in the production of ammonia), creating faster reaction rates. Flow reactors enable excellent reaction selectivity. The rapid diffusion mixing avoids the issues found in batch reactors. The high surface area to volume ratio of the catalytic nanorod arrays and their plasmonic properties enables instantaneous local heating and therefore ultimate temperature control, resulting in higher yields and higher selectivity.

In catalytic plasmonic nanomaterial enabled flow reactors, heat transfer is intensified, because the area to volume ratio is large, hence endothermic and exothermic reactions can be easily and consistently regulated. The steep temperature gradient provides efficient control over reaction time, and at the same time prevents further reactions of products that are out of the heated zone.

The flow reactor 10 can be employed in series with reaction products exiting one catalytic zone to be flowed into another catalytic zone, allowing multi-step synthesis using consecutive reactions, without the need for separation steps in between. This can be especially beneficial if intermediate compounds are unstable, toxic, or sensitive to air, since they exist only briefly, and in very small quantities. Flow reactors allow easy coupling to separation and analysis in, for example, gas chromatograph-mass spectrometer (GCMS), as well as to in-line FTIR. Flow chemistry facilitates reaction conditions not possible in batch such as a very short contact time, and control of contact time by adjusting the flow rate. Such control results in cleaner reaction and minimizes side product and costly separation and purification.

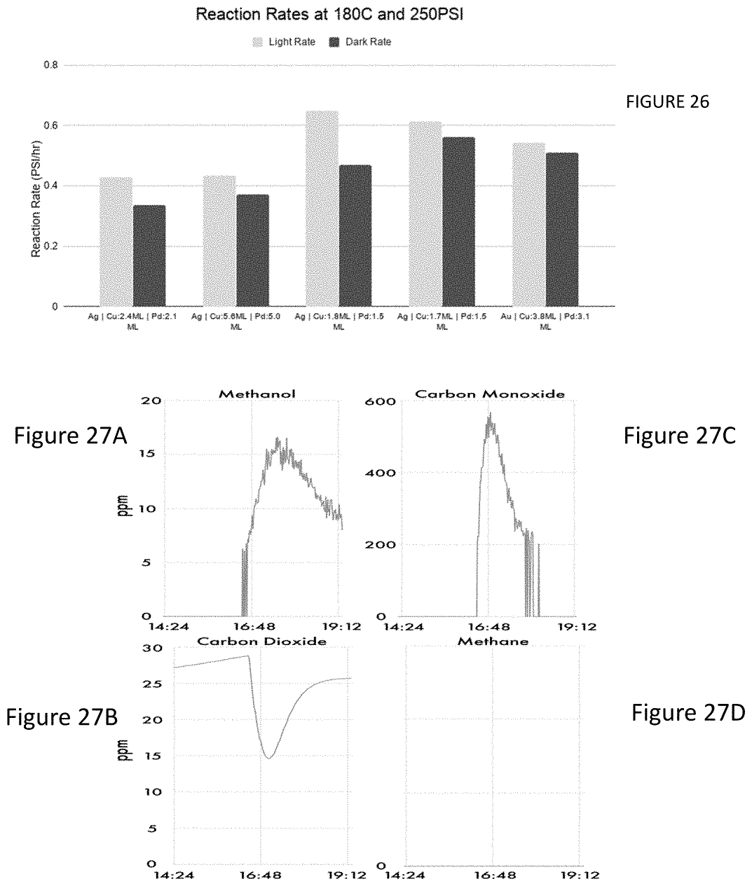

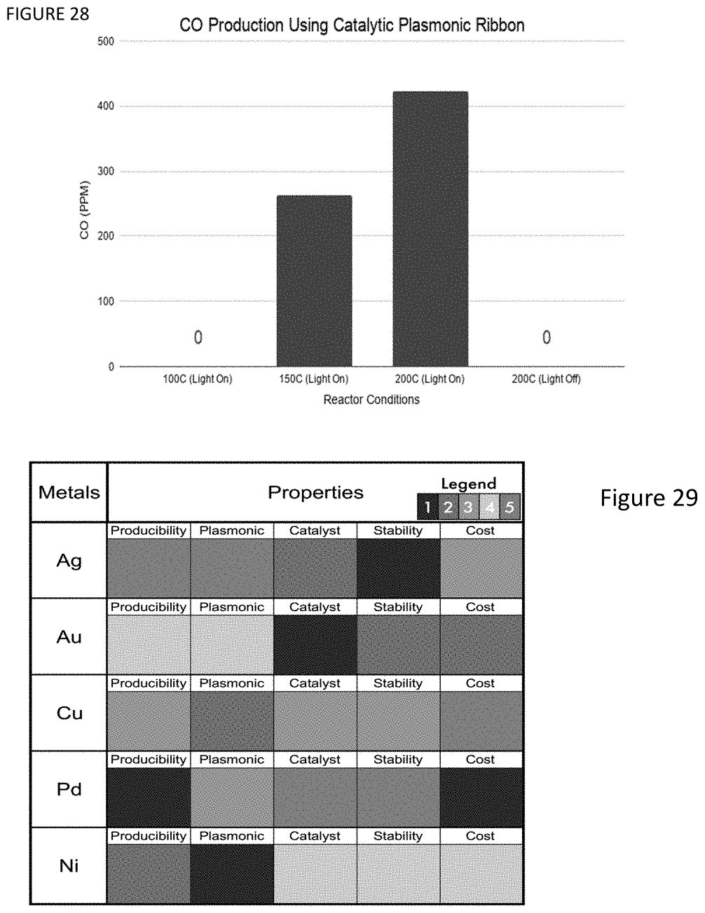

The injection of hot carriers lowers the threshold energy, so it is anticipated methanol production via photocatalytic pathways will occur at ambient temperature and will be measured as a function of input H₂ and CO₂ gas pressure. Under illumination, a pressure decrease indicates that the reaction is occurring. FIG. 26 shows the difference in reaction rates (pressure) for no illumination (dark) and illuminated (light) Cu—Pd coated silver nanorod arrays reacting with a 2:1 H₂ to CO₂ using the reactor shown in FIG. 28. The reaction products can further be analyzed using an in-line gas sampling loop to a Gas Chromatograph or gas analyzer with thermal conductivity meter. The reaction products are analyzed as a function of inlet pressure, illumination intensity, and temperature.

### EXAMPLE 4

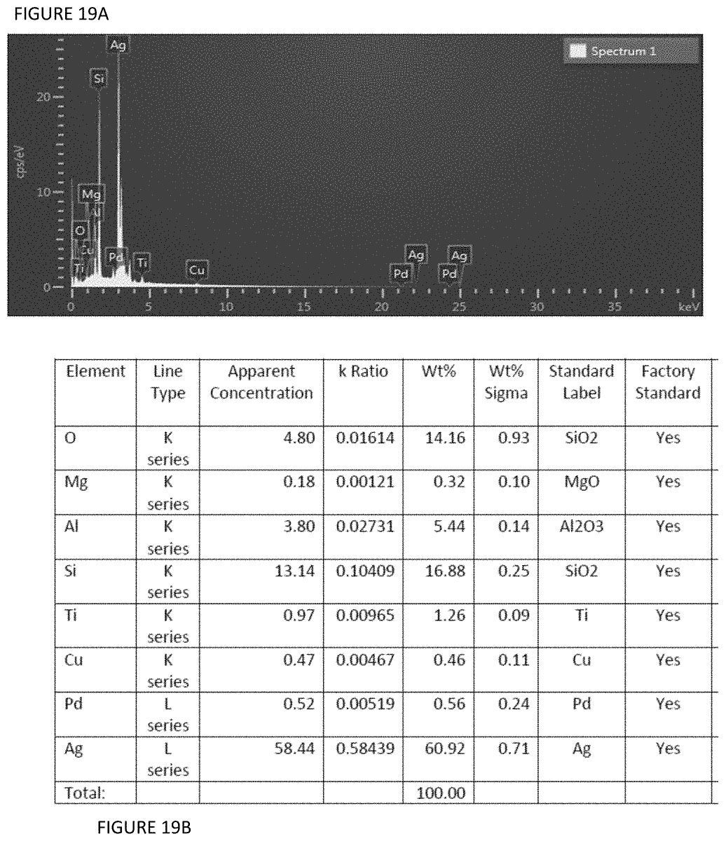

A physical vapor deposition (PVD) system was used to simultaneously co-deposit copper and palladium via two-target sputtering using independently controlled rf and dc power sources. This technique allows the operator to control the amounts of each metal deposited and was calibrated with the goal of yielding a 1:1 Cu:Pd bimetal coating on the as produced silver nanorod surfaces. The calibration process first involves tuning the deposition parameters using glass slide substrates prior to utilizing catalytic plasmonic nanomaterial samples. The results obtained by varying the deposition parameters of temperature, pressure, target to source spacing, relative power, and deposition time on catalytic plasmonic nanomaterial and planar glass witness substrates were analyzed using Energy Dispersive X-Ray Emission Spectroscopy (EDX) to directly measure the compositional analysis. These data are presented in FIG. 19A-19B, which primarily demonstrates that co-deposition conditions were obtained where equal amounts of Cu and Pd were co-deposited onto the nanorod surfaces. All the other elements detected agree with the compositional analysis of the glass substrate and the coatings that were applied.

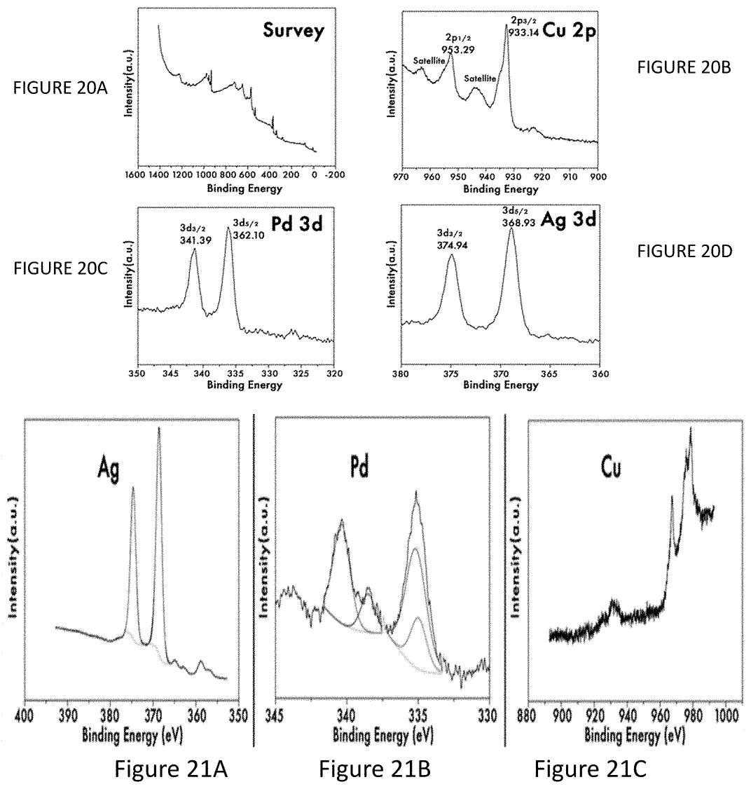

XPS was used to probe the electronic states of the surface elements on the catalytic plasmonic nanomaterial samples. These data are presented in FIGS. 20A-20D which shows spectra for the system (survey), and individual spectra measured for Ag, Cu, and Pd. Both Pd and Ag are observed to be in an elemental metal state, while the Cu is partially oxidized to the 2+ state.

The summary table for co-ED results are shown in TABLE 1. All ED baths contain a reducible metal salt, reducing agent (RA) and stabilizer in pH-adjusted water. Baths and ED conditions that have been tested for deposition of Pd²⁺ and Cu²⁺ salts are shown below. The optimized bath is shown at the bottom in the gray background.

TABLE 1
Summary of the Electroless Deposition Parameters used to optimize
the co-deposition of controlled Cu—Pd monolayers on silver.

| | | | [RA]/[stabilizer]/ | | | % | |
|---|---|---|---|---|---|---|---|
| metal source | RA | stabilizer | [metal] | pH | Temp | deposited | θ_{M} on Ag |
| Cu(NO₃)₂ | DMAB | EDTA | 5/1/1 | 9 | 70 C. | 31% | 0.31 |
| Na₂PdCl₄ | DMAB | EDTA | 5/1/1 | 9 | 70 C. | 42% | 0.42 |
| Cu(NO₃)₂ | DMAB | EDTA | 2/1/1 | 7 | 70 C. | 29% | 0.29 |
| Na₂PdCl₄ | DMAB | EDTA | 2/1/1 | 7 | 70 C. | 28% | 0.28 |
| Cu(NO₃)₂ | HCHO | EDTA | 5/1/1 | 10 | 70 C. | 14% | 0.14 |
| Na₂PdCl₄ | HCHO | EDTA | 5/1/1 | 10 | 70 C. | 4.6% | 0.05 |
| Cu(NO₃)₂ | HCHO | EDTA | 5/1/1 | 12 | 70 C. | 35% | 0.35 |
| Na₂PdCl₄ | HCHO | EDTA | 5/1/1 | 12 | 70 C. | 20% | 0.2 |
| Cu(NO₃)₂ | HCHO | EDTA | 10/1/1 | 10 | 70 C. | 9% | 0.09 |
| Na₂PdCl₄ | HCHO | EDTA | 10/1/1 | 10 | 70 C. | 4.4% | 0.04 |
| Cu(NO₃)₂ | HCHO | EDTA | 10/1/1 | 12 | 70 C. | 80% | 0.8 |
| Na₂PdCl₄ | HCHO | EDTA | 10/1/1 | 12 | 70 C. | 40% | 0.4 |
| Cu(NO₃)₂ | N₂H₄ | EDTA | 5/1/1 | 10 | 25 C. | 55% | 0.55 |
| Na₂PdCl₄ | N₂H₄ | EDTA | 5/1/1 | 10 | 25 C. | 48% | 0.48 |
| Cu(NO₃)₂ | N₂H₄ | EN | 5/2/1 | 9 | 25 C. | 100% | 1.0 |
| Pd(NH₃)₄Cl₂ | N₂H₄ | EN | 5/1/1 | 9 | 25 C. | 98% | 1.0 |

DMAB = dimethylamine borane;
EDTA = ethylenediaminetetraacetic acid;
EN = ethylenediamine

TABLE 1 shows near complete deposition of Cu²⁺ and Pd²⁺ at 25° C. using hydrazine (N₂H₄) as the reducing agent and ethylenediamine (EN) as the stabilizer has been achieved. The ED bath is thermodynamically unstable, but kinetically stable in the absence of a catalytic surface, which is Ag in this case. Before doing co-ED on the Ag base catalyst, all baths were checked for thermal stability (no catalyst present in the bath) to ensure only deposition of Cu and Pd on the Ag surface by ED, rather than thermal reduction of Cu²⁺ and Pd²⁺ by the reducing agent (N₂H₄) in the ED bath. By using EN as a stabilizer for Cu(NO₃)₂ and Pd(NH₃)₄Cl₂, all ED baths exhibited good thermal stability of both Cu²⁺ and Pd²⁺ salts.

Using the co-ED method where each metal salt solution and the reducing agent were added by syringe pumps (three in all), one theoretical monolayer (ML) of Cu and 1ML of Pd salts were added to the 5% Ag/SiO₂ compound, where the Ag surface site concentration had been measured by both XRD peak broadening and selective O₂—H₂ chemisorption. Dispersion values are typically lower for chemisorption than XRD peak broadening and this study was no different (0.035 vs 0.092, respectively). A chemisorption value of 9.8×10¹⁸ surface Ag sites/g cat was directly measured and not inferred from x-ray line broadening of the Ag (111) peak observed at 2θ=38.5° as determined by application of the Debye-Scherrer equation.

XPS was used to probe the electronic states of the surface elements on the ED coated catalytic plasmonic nanomaterial samples. These data are presented in FIGS. 21A-21C, which show spectra for the system (survey), and individual spectra measured for Ag, Cu, and Pd. Large amounts of silver are observed in the sample, existing in the form of elemental metal Ag. Trace amount of Pd 3d signal is seen, indicating both Pd (II) and Pd (0) states exist. Cu peaks are very weak and appear to be oxidized to Cu (II).

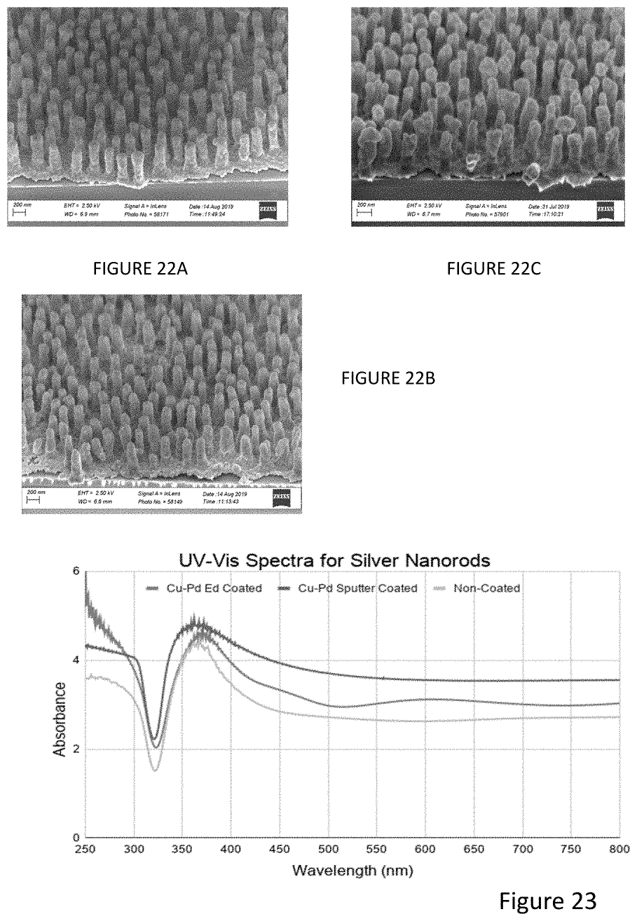

The results of applying a Cu—Pd bimetallic coating to silver nanorod arrays were initially assessed using SEM imaging, as shown in FIGS. 22A-22C. These images reveal that the morphology of the structures is altered significantly by the sputter coating, which adds material to the upper portions of the nanorods, while the ED coating appears uniform and difficult to observe compared to the pristine nanorods.

The UV-Vis spectra of the samples shown in FIGS. 22A-22C were measured to quantify any alterations in the plasmonic absorption of the silver plasmonic nanomaterial samples that resulted from the respective ED and sputter deposition processes employed to catalytically activate the material. The results are presented in FIG. 23 that shows the peak plasmon resonance wavelength and overall absorbance intensity is not significantly altered or damped by the application of the coatings.

### EXAMPLE 5

### Design, Modeling, and Fabrication of Advanced Photocatalytic Reactors for Chemical Synthesis

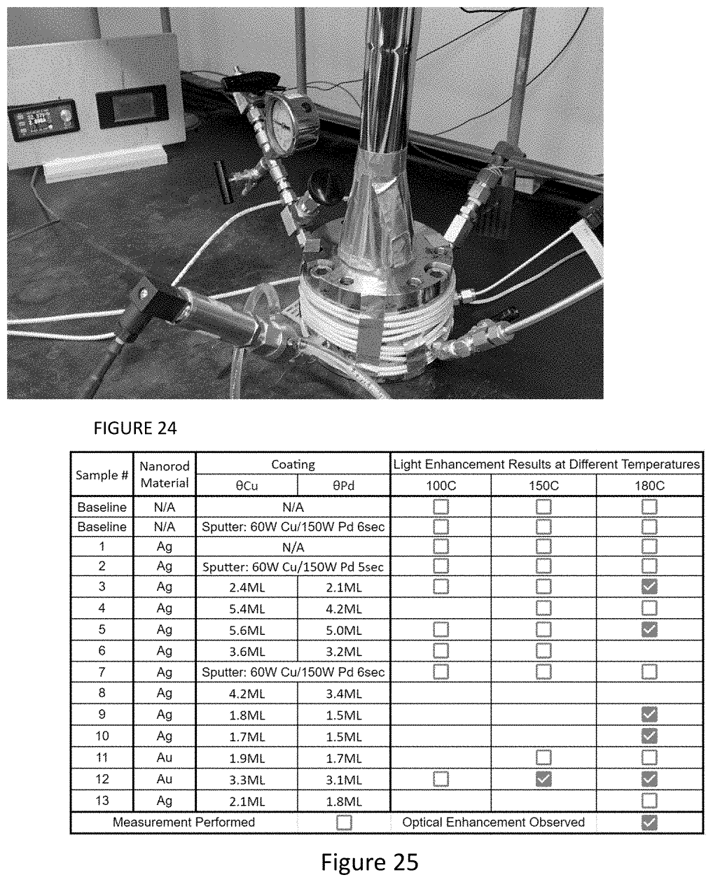

A batch photoreactor similar to that in EXAMPLE 3 was customized by Parr Instruments to evaluate the catalytic plasmonic nanomaterials produced, shown in FIG. 24. The reactor is made of T316 steel, and is rated to 600 psi (41 atm.) and 150° C. The test chamber is cylindrical with an I.D. of 2.380″ (accommodating a photocatalytic plasmonic nanomaterial of O.D. 2″), with 1.400″ depth, and a 100 mL volume. A fused silica window with 2.380″ diameter viewing area assures the photocatalyst's full illumination. Two external valves and fittings allow the option of using the reactor in either batch or flow reaction modes. A thermocouple allows continuous temperature recording. The device is equipped with a 4838 controller with pressure display module, an Omega transducer with +/−0.05% accuracy, 0-750 psi (absolute pressure), a cooling adapter, and a computer connection. SpecView software for data logging of temperature and pressure to 000.1 resolution allows continuous recording of gas evolution (increased pressure), and hence kinetics studies. An LED irradiation source is positioned above the window to provide controlled sample illumination at a given wavelength. This reactor is used in batch mode and gas samples can be extracted from a valve sealed pressure port.

Measurements are performed by filling the reactor with a 3:1 ratio of H₂ and CO₂ at 250 psi and 180° C. to ensure the complete reduction of any metal oxide layer that may have formed on the surface. The gas mixture was then replaced with a fresh mixture and the change in pressure was monitored at a given temperature without irradiation for 24 hours. Thereafter, the gas was replaced again with a fresh mixture, and the pressure was monitored at same temperature under LED illumination. The LED used for silver samples was 365 nm and for gold 560 nm at power levels of ˜1000 w/m².

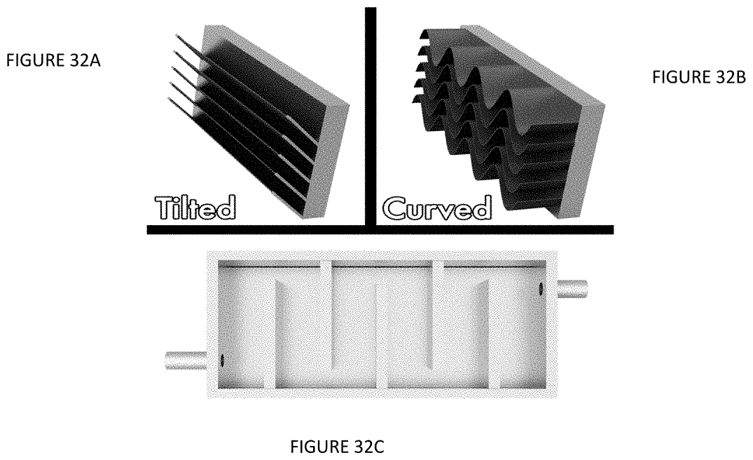

FIG. 25 shows a table of the samples measured. Samples were measured in light and dark reactor conditions. Those measurements with higher reaction rates under illumination are marked. All measurements had greater or equal rates under illumination compared to dark data and were performed at P=250 psi. The results of kinetic measurements are shown in FIG. 32, which shows the difference between light and dark reaction rates for five ED coated samples (4 silver and 1 gold), which also shows the amounts of Cu and Pd deposited. Optical rate enhancement for sputter coated samples was not observed.

Data from an optical flow reactor system are shown in FIGS. 27A-27D, which shows the products obtained from a batch measurement performed at 200° C. The vessel was pressurized with 1:3 CO₂:H₂ and illuminated at 365 nm and 50 W and left under illumination for ˜8 hours before flushing out the chamber with source gases. There was an immediate increase in CO and MeOH product gases detected once the chamber was opened, which are observed to peaked and then decrease over a >2-hour time span. This is due to the mixing and diffusion with the slow flow rates and the retention time for the gases. The peak time for CO and dip time for CO₂ is the same. The peak for methanol occurred later possibly due to it being able to ‘wet’ the reactor surface or get trapped elsewhere in liquid form. The results showed some significant CO formation, but relatively low overall (peak ˜550ppm). The methanol formation was low (peak ˜16 ppm).

FIG. 28 shows the measured the production of CO as a function of temperature on an optical reactor. These data were obtained at reactor pressures of 20 psi in batch mode. The system was fully flushed with source gas between each batch run, which was performed under illumination at 100° C., 150° C., 200° C., where the 200° C. measurement was performed with and without illumination. The results are presented in FIG. 28 that shows CO production is very dependent on temperature and light under these conditions. At 100° C., no CO is produced, while it turns on at 150° C. and increases in level at 200° C. Then, when the illumination is turned off, the CO production returns to zero.

The bench top reactor results can be input to computer simulations to achieve the best distribution of catalytic material in the reactor for optimal energy efficiency and output. A primary goal is using low-cost materials and processes to fabricate the elements of the system. Optical reactors that convert a maximum number of photons into a maximum number of methanol molecules at the highest cost to product value ratio are sought. Catalytic reactors must be robust, have long lifetimes, and high product yields to be deployed commercially. Solar capacity dictates lower operating temperatures and pressures than conventionally used. These characteristics can be analyzed using computer modeling results concurrently with engineering to cyclically determine the most readily attainable conditions to rapidly achieve the next level design evolution. Thus, reactor designs can begin with planar catalytic plasmonic nanomaterial housings. A low-profile substrate geometry and high surface area afforded through nanoengineering can be used to configure a system with maximum light absorption combined with reactant and product flow that will minimize dead zones and fully utilize the unique capabilities provided by format. The optimal internal reactor configuration for the catalytic plasmonic nanomaterials can be identified by quantitatively measuring various scenarios that are supported by the computer modeling work as displayed in FIGS. 32A and 32B. FIG. 32A shows tilted catalytic plasmonic nanomaterials, FIG. 32B shows curved catalytic plasmonic nanomaterials, and FIG. 32C shows a baffled reactor.

Once scaled up, the reactors can be used with flexible glass as well as solar power. FIGS. 31A-31B show tube reactor designs with baffles of catalytic plasmonic nanomaterials, and FIGS. 31C-31D show the reactors coupled with solar collectors and deployed in large area arrays.

### EXAMPLE 6

### Establishing a Baseline for Catalytic and Plasmonic Materials Used in Fabrication

A matrix of potential material candidates from which catalytic plasmonic nanomaterials can be made from can be evaluated for merit of the various properties desirable to this technology. Baseline measurements are performed on nanorod arrays made of Ag, Au, Cu, Pd, and Ni to determine the initial structure, initial spectra, changes upon heating, changes upon illumination, and relevant adsorbate properties. An evaluation matrix is shown in FIG. 29, which rates the different materials using a scale of 1 (unfavorable) to 5 (favorable), for the known properties of compatibility in: Producibility (fabrication) of catalytic plasmonic nanomaterials, the magnitude and useful characteristics of its base plasmonic effect, its effectiveness as a catalyst, its likely long-term stability in photocatalytic synthesis, and its cost. A matrix like this is useful in designing plasmonic catalyst for targeted and industrial synthesis

Key to the analysis is the catalytic capacity to dissociate CO₂ to CO and an oxygen atom, which is observed on the surface of a plasmonic Ag nanoparticles. The different possible routes for the reduction of CO to methanol or methane on the Ag surface are energy dependent, with all elementary reactions for CH₄ having a larger activation energy barrier than those leading to CH₃OH formation, suggesting that lower working temperatures can be used to minimize CH₄ production. Another possible mechanism is CO₂ reduction on Ag nanoparticles without dissociation.Error! Bookmark not defined.

Silver is the best plasmonic metal, however, thermal instability has been observed. The use of Ag—Pd alloys can be examined. While bulk Ag melts at 961.8° C., Ag—Pd alloys melt at 1155-1250° C., depending on composition. Gold melts at 1,064° C., and Au nanorod arrays remained stable above 180° C. Adding Pd to Ag will also extended the plasmonic bands toward NIR, with optimal response at 25.8% Pd. The Ag—Pd alloy systems consists of a homogenous solid solution phase over its entire composition range that is comprised of Ag-rich and Pd-rich nanoclusters that may benefit the Ag plasmonic and Pd catalytic properties. Ag—Pd alloys can be electrodeposited from a chloride rich solution with acidic pH, or a plating solution resulting in Ag-rich Ag—Pd films consisting of PdCl₂, AgNO₃, HBr, and HNO₂. Highly acidic solutions are not compatible with the Al₂O₃ templating process. High Ag percentages were reported in ammonia solutions, with pH11.5 at room temperature (22° C.). For the template process, the pH can be adjusted to 9-9.5. Plated Ag—Pd alloy's with Pd concentration of 15-25% have been achieved under the following conditions (concentrations in M): Pd 0.15-0.20; Ag 0.02-0.03; Trilon B 0.12-0.20; (NH4)2CO3 0.10-0.20; NH4OH 0.25-0.50; pH 9.0-9.5; temp. 20-40°.

A series of catalytic plasmonic nanomaterials can be fabricated from the pure metals shown in FIG. 29 using the methods described in FIGS. 2A-2D. The structure is assessed by SEM, while the plasmonic response is measured using UV-vis spectroscopy.

Four nanorod structural designs that encompass methods readily available are presented in FIG. 30. An attractive geometry to combine plasmonic and catalytic properties is to effectively position them a neighbor in an antenna-reactor geometry. Such systems consist of (at least) two separated parts: a plasmonic antenna which collects light, and a catalytic reactor which facilitates the reaction. In the layered structure, the plasmonic antenna and a catalytic reactor interface and strong interlayer coupling expected with both “hot” electrons and holes to interact with surface adsorbates and reduce the activation energies to reduce CO2 to CH3OH.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method for producing plasmonic nanomaterials that are catalytically or photocatalytically active, including the steps of:
fabricating plasmonic nanostructures on substrates coated with a conductive seed layer and then a nanoporous template and using electrodeposition into structure of the nanoporous template and forming a plurality of nanorods in an array, wherein the nanorods are made from materials chosen from the group consisting of materials that are plasmonic and/or catalytic, and materials that are catalytically activated by depositing pure elemental metals, alloys, or alternating layers of different metals or alloys; and
producing catalytic plasmonic nanomaterials.

2. The method of claim 1 wherein the nanoporous template is removed exposing a freestanding array of vertically aligned nanorods attached to an underlying substrate.

3. The method of claim 1, wherein the nanorods include catalytic surface materials applied by a step chosen from the group consisting of a) capping or coating nanorod arrays using electrodeposition, b) capping or coating nanorods using electroless chemical deposition, c) capping or coating nanorods with catalytic coatings using physical vapor deposition methods, and d) by coating or covering nanorods with catalytic material using wet chemistry applications, wherein the plasmonic nanomaterials are vertically aligned arrays of nanorods with one radial end of the nanorod attached through a conductive layer to a substrate.

4. The method of claim 1, wherein the plasmonic nanomaterials are formed on substrates having a format chosen from the group consisting of ribbons, sheets, or rolls of flexible glass, a ribbon, sheet or roll of polymeric materials, a foil, a thread produced on glass, polymer, or metal fibers, in a rigid planar design that is insulating or conducting, and on the inner or outer surfaces of a tube.

5. The method of claim 1, wherein flexible substrates are used and the fabrication is performed in a continuous or roll-to-roll format.

6. The method of claim 1, wherein rigid substrates are used and batch processed using an immersible electrochemical cell.

7. The method of claim 1, wherein the conductive seed layer is made of a material chosen from the group consisting of silver, gold, aluminum, tungsten, nickel, palladium, cobalt, molybdenum, platinum, copper, zinc, iron iridium, indium tin oxide, aluminum-doped zinc oxide, poly(3,4-ethylenedioxythiophene), carbon nanotubes, and graphene.

8. The method of claim 1, wherein the nanorods are made of a material chosen from the group consisting of silver, gold, aluminum, copper, cobalt, chromium, iron, molybdenum, manganese, indium, nickel, palladium, platinum, rhodium, tantalum, titanium, titanium nitride, tungsten, silicon, tin, zirconium nitride, zinc, iridium, alloys thereof, nitrides thereof, and oxides thereof.

9. The method of claim 1, wherein a material used for capping or coating the nanorods is chosen from the group consisting of palladium, nickel, platinum, silver, titanium, gold, ruthenium, rhodium, iridium, nickel, iron, chromium, zinc, copper, Al₂O₃, CuO, Fe₂O₃, TiO₂, SnO₂, V₂O₅, WO₃, ZrO₂, ZnO, Cu/ZnO and Cu/ZnO₂, MnOₓ/m-Co₃O₄, In₂O₃/ZrO₂, and Pd—Zn alloys.

10. The method of claim 1, further including the step of producing a layered nanorod array of plasmonic and/or catalytic layers constituting nanostructures by alternating depositions between electroplating baths of two or more metals or alloys.

11. The method of claim 1, wherein said step of capping or coating nanorod arrays uses electrodeposition and is further defined as a step chosen from the group consisting of producing bimetallic nanocaps on the nanorods, and fully coating the nanorods resulting in a core-shell nanostructural formation.

12. The method of claim 1, wherein said step of capping or coating nanorod arrays uses electroless deposition and is further defined as a step chosen from the group consisting of producing bimetallic nanocaps on the nanorods, and fully coating the nanorods resulting in a core-shell nanostructural formation.

13. The method of claim 1, wherein the coating is chosen from the group consisting of a sputter coating, thermal evaporation, electron beam evaporation, atomic layer deposition, and chemical vapor deposition.

14. The method of claim 1, wherein the nanorods have dimensions of about 50-150 nm diameters and about 400-2000 nm lengths with center-to-center spacing of about 75-300 nm.

15. The method of claim 1, wherein when the nanorods are illuminated at or near their plasmon resonance wavelength.

16. Catalytic plasmonic nanomaterials made from the method of claim 1.

17. An optical reactor device that utilizes plasmonic catalytic nanomaterials for photocatalytic synthesis of fuels and chemicals including methanol, ethylene, or ammonia, comprising:
a chemical reaction chamber containing a catalytic plasmonic nanomaterial, said chemical reaction chamber including a gas distribution manifold for flowing gas containing reactive components over said catalytic plasmonic nanomaterial and a gas collection manifold for collecting synthesized gas products, wherein said chemical reaction chamber includes a mechanism of providing optical energy to said catalytic plasmonic nanomaterial through illumination and provides constant temperature control of the chemical reaction chamber.

18. The optical flow-reactor device of claim 17, wherein the mechanism of providing optical energy is further defined as a LED array.

19. The optical flow-reactor device of claim 17, where an LED wavelength matches a plasmon resonance wavelength of the catalytic plasmonic nanomaterial.

20. The optical flow-reactor device of claim 17, wherein the catalytic plasmonic nanomaterial includes vertically aligned arrays of nanorods with one radial end of a nanorod attached through a conductive layer to a substrate.

21. The optical flow-reactor device of claim 20, wherein the substrate is glass.

22. The optical flow-reactor device of claim 17, wherein the conductive layer is made of a material chosen from the group consisting of silver, gold, aluminum, tungsten, nickel, palladium, cobalt, molybdenum, platinum, copper, zinc, iron iridium, indium tin oxide, aluminum-doped zinc oxide, poly(3,4-ethylenedioxythiophene), carbon nanotubes, and graphene.

23. The optical flow-reactor device of claim 17, wherein the nanorods are made of a material chosen from the group consisting of silver, gold, aluminum, copper, cobalt, chromium, iron, molybdenum, manganese, indium, nickel, palladium, platinum, rhodium, tantalum, titanium, titanium nitride, tungsten, silicon, tin, zirconium nitride, zinc, iridium, alloys thereof, nitrides thereof, and oxides thereof.

24. The optical flow-reactor device of claim 14, wherein a source of optical energy is solar power.

25. The optical flow-reactor device of claim 17, wherein said optical flow-reactor device is mounted in a concentrating solar collector.

26. The optical flow-reactor device of claim 17, wherein said catalytic plasmonic nanomaterial is arranged in a design chosen from the group consisting of baffles, a tilted design, and a curved design.

27. A method of photocatalytic synthesis of chemicals and fuels including methanol and ammonia, including the steps of:
using catalytic plasmonic nanomaterial to convert CO₂ and H₂ to methanol, CO₂ and CH₄ to methanol, and N₂ and H₂ to ammonia using optical power.

28. The method of claim 27, wherein reactants input into a reactor to produce the methanol and ammonia are chosen from the group consisting of CO₂, H₂, H₂O, and CH₄.

29. The method of claim 27, further including the steps of photo-absorbing to activate the catalytic plasmonic nanomaterial, generating heat and energetic charge carriers from the activated catalytic plasmonic nanomaterial, thereby driving catalytic reaction between catalyst deposited on the catalytic plasmonic nanomaterial and reactants introduced into the reactor, and producing chemicals and fuels.

30. The method of claim 27, wherein said step of using optical power is further defined as providing optical energy to the catalytic plasmonic nanomaterial through an LED array in an optical flow-reactor device.

31. The method of claim 30, wherein the LED is tuned to a plasmon resonance wavelength of the catalytic plasmonic nanomaterial.

32. Methanol and ammonia made by the method of claim 27.

33. A method of synthesizing useful chemicals from greenhouse gases such as CO₂ and CH₄ and waste gases that are used as sources in the synthesis of chemicals and fuels, including the step of:
using catalytic plasmonic nanomaterial to convert greenhouse gases to useful chemicals using optical power.

34. A method of making plasmonic nanomaterial, including the step of:
forming plasmonic nanorods on a flexible substrate.

35. A method of producing chemicals, including the steps of:
stimulating plasmons in catalytic plasmonic nanomaterials with photons in a plasma catalytic reactor; and
producing chemicals.

36. The method of claim 35, wherein optical excitation from plasma in the catalytic plasmonic nanomaterials and excited molecules, atoms, ions, electrons, radicals, and photons in the plasma work together to break chemical bonds of stable molecules and interact with the catalytic plasmonic nanomaterials to produce chemicals.

37. A hybrid plasma-plasmonic reactor device that utilizes plasmonic catalytic nanorod arrays for synthesis of fuels and chemicals including methanol or ammonia, comprising:
a reaction chamber containing a first adjustable disc electrode having first catalytic plasmonic nanomaterial layer thereon and a second adjustable disc electrode having second catalytic plasmonic nanomaterial layer thereon, said reaction chamber including a gas inlet for flowing gas containing reactive components over said first and second catalytic plasmonic nanomaterials and a gas outlet for collecting synthesized gas products, wherein said first and second catalytic plasmonic nanomaterial layers ignite a plasma from gas introduced into said reaction chamber and synthesize fuels and chemicals.
